(19) United States
(12) Patent Application Publication
(10) Pub. No.: US 2002/0006613 A1
(43) Pub. Date: Jan. 17, 2002

(54) METHODS AND COMPOSITIONS FOR THE IDENTIFICATION AND ASSESSMENT OF CANCER THERAPIES

(76) Inventors: ANDREW W. SHYJAN, NAHANT, MA (US); CHRISTOPHE VAN HUFFEL, Brussels (BE)

Correspondence Address: LAHIVE & COCKFIELD, 28 STATE STREET, BOSTON, MA, 02109, US

(21) Appl. No.: 09/374,554

(22) Filed: Aug. 13, 1999

Related U.S. Application Data
(63) Continuation-in-part of application No. 09/134,411, filed on Aug. 13, 1998.
(60) Provisional application No. 60/064,687, filed on Nov. 5, 1997.
(60) Provisional application No. 60/096,666, filed on Aug. 13, 1998.
(60) Provisional application No. 60/096,662, filed on Aug. 13, 1998.
(60) Provisional application No. 60/037,921, filed on Feb. 12, 1997.

(51) Int. Cl. C07H 21/02; C07H 21/04; C12Q 1/68

(52) U.S. Cl. 435/6; 536/23.1

(57) ABSTRACT

The present invention is directed to the identification of markers that can be used to determine whether cancer cells are sensitive or resistant to a therapeutic agent. The present invention is also directed to the identification of therapeutic targets. Nucleic acid arrays were used to determine the level of expression of approximately 6500 nucleic acid sequences (genes) found in 54 different solid tumor cancer cell lines selected from the NCI 60 cancer cell line series. After the level of expression was determined for each of the 6500 genes in each of the 54 cancer cell lines, statistical analysis was used to identify genes whose expression correlated with sensitivity or resistance to any one of 171 different anti-cancer compounds. Expression analysis was also used to identify genes associated with resistance or sensitivity to TAXOL in a number of cancer cell lines, clinical samples, and a human mammary epithelial cell primary cell line. The invention features a number of “sensitivity genes.” These are genes that are expressed in most or all cell lines that are sensitive to treatment with an agent and which are not expressed (or are expressed at a rather low level) in cells that are resistant to treatment with that agent. The invention also features a number of “resistance genes.” These are genes that are expressed in most or all cell lines that are resistant to treatment with an agent and which are not expressed (or are expressed at a rather low level) in cells that are sensitive to treatment with that agent.

### RELATED APPLICATION INFORMATION

This application claims priority from U.S. Ser. No. 09/322,864, filed May 28, 1999, which claims priority from U.S. Ser. No. 09/233,611, filed Jan. 19, 1999, which claims priority from provisional application serial No. 60/105,968, filed Oct. 28, 1998, provisional application serial No. 60/079,399, filed Mar. 26, 1998, and provisional application serial No. 60/071,940, filed Jan. 20, 1998. The contents of each of the aforementioned applications are expressly incorporated by reference.

### BACKGROUND OF THE INVENTION

Cancers can be viewed as a breakdown in the communication between tumor cells and their environment, including their normal neighboring cells. Growth-stimulatory and growth-inhibitory signals are routinely exchanged between cells within a tissue. Normally, cells do not divide in the absence of stimulatory signals or in the presence of inhibitory signals. In a cancerous or neoplastic state, a cell acquires the ability to “override” these signals and to proliferate under conditions in which a normal cell would not.

In general, tumor cells must acquire a number of distinct aberrant traits in order to proliferate in an abnormal manner. Reflecting this requirement is the fact that the genomes of certain well-studied tumors carry several different independently altered genes, including activated oncogenes and inactivated tumor suppressor genes. In addition to abnormal cell proliferation, cells must acquire several other traits for tumor progression to occur. For example, early on in tumor progression, cells must evade the host immune system. Further, as tumor mass increases, the tumor must acquire vasculature to supply nourishment and remove metabolic waste. Additionally, cells must acquire an ability to invade adjacent tissue. In many cases cells ultimately acquire the capacity to metastasize to distant sites.

It is apparent that the complex process of tumor development and growth must involve multiple gene products. It is therefore important to define the role of specific genes involved in tumor development and growth and identify those genes and gene products that can serve as targets for the diagnosis, prevention and treatment of cancers.

In the realm of cancer therapy it often happens that a therapeutic agent that is initially effective for a given patient becomes, overtime, ineffective or less effective for that patient. The very same therapeutic agent may continue to be effective over a long period of time for a different patient. Further, a therapeutic agent that is effective, at least initially, for some patients can be completely ineffective or even harmful for other patients. Accordingly, it would be useful to identify genes and/or gene products that represent prognostic markers with respect to a given therapeutic agent or class of therapeutic agents. It then may be possible to determine which patients will benefit from particular therapeutic regimen and, importantly, determine when, if ever, the therapeutic regime begins to lose its effectiveness for a given patient. The ability to make such predictions would make it possible to discontinue a therapeutic regime that has lost its effectiveness well before its loss of effectiveness becomes apparent by conventional measures

### SUMMARY OF THE INVENTION

The present invention is directed to the identification of markers that can be used to determine whether cancer cells are sensitive or resistant to a therapeutic agent. The present invention is also directed to the identification of therapeutic targets.

The invention features a number of “sensitivity genes.” These are genes that are expressed in most or all cell lines that are sensitive to treatment with an agent and which are not expressed (or are expressed at a rather low level) in cells that are resistant to treatment with that agent. The invention also features a number of “resistance genes.” These are genes that are expressed in most or all cell lines that are resistant to treatment with an agent and which are not expressed (or are expressed at a rather low level) in cells that are sensitive to treatment with that agent.

Nucleic acid arrays were used to determine the level of expression of approximately 6500 nucleic acid sequences (Table 4) found in 54 different solid tumor cancer cell lines (Table 5) selected from the NCI 60 cancer cell line series. After the level of expression was determined for each of the 6500 genes in each of the cancer cell lines, statistical analysis was used to identify genes whose expression correlated with sensitivity or resistance to any one of 171 different anti-cancer compounds (Table 3). The sensitivity and resistance genes identified in this study are presented in Tables 1, 2A, and 2B.

Nucleic acid arrays were also used to determine the level of expression of approximately 6500 murine nucleic acid sequences in a cyclophosphamide resistant murine epithelial tumor cell line and in a cisplatin resistant murine epithelial tumor cell line. This analysis led to the identification of genes that are expressed at a higher level in the cyclophosphamide resistant cell line than in the parent cell line from which the resistant line was derived (Table 7A), genes that are expressed at a lower level in the cyclophosphamide resistant cell line than in the parent cell line from which the resistant line was derived (Table 7B), genes that are expressed at a higher level in the cisplatin resistant cell line than in the parent cell line from which the resistant line was derived (Table 7C), and genes that are expressed at a lower level in the cisplatin resistant cell line than the parent cell line from which the resistant line was derived (Table 7D). The resistance genes identified in this study are presented in Tables 7A-7D.

Nucleic acid arrays were also used to determine the level of expression of approximately 6500 nucleic acid sequences in selected relatively TAXOL resistant and in selected relatively TAXOL sensitive solid tumor cell lines from the NCI 60 cancer cell line series. This analysis led to the identification of resistance genes that are relatively highly expressed in relatively TAXOL resistant cell lines (Tables 8A, 9A, 9B, 9C, and 9D). This study also led to the identification of sensitivity genes that are relatively highly expressed in relatively TAXOL sensitive lines (Table 8B).

In another study, nucleic acid arrays were used to determine the level of expression of approximately 6500 nucleic acid sequences in a relatively TAXOL resistant human mammary epithelial cell primary cell line (HMEC) and in a relatively TAXOL sensitive breast cancer cell line (MDA-435) in the presence of TAXOL. This analysis led to the identification of genes that are relatively highly expressed in the relatively TAXOL resistant human mammary epithelial cell primary cell line compared to the relatively TAXOL sensitive breast cancer cell line (Table 10A) and genes that are relatively highly expressed in the relatively TAXOL sensitive breast cancer cell line compared the TAXOL resistant human mammary epithelial cell primary cell line (Table 10B). Thus, Table 10A present resistance genes, and Table 10B presents sensitivity genes.

In yet another study, nucleic acid arrays were used to determine the level of expression of approximately 20,000 nucleic acid sequences in clinical samples obtained from patients whose ovarian cancer appeared to respond to TAXOL/cisplatin combination therapy over an initial six month period (“TAXOL/cisplatin sensitive clinical samples”) and in clinical samples obtained from patients whose ovarian cancer appeared to respond poorly to TAXOL/cisplatin combined therapy over an initial six month period (“TAXOL/cisplatin resistant clinical samples”).

This analysis led to the identification of genes that are expressed at a relatively high level in the TAXOL/cisplatin resistant clinical samples compared to the TAXOL/cisplatin sensitive clinical samples (Table 11A) and genes that are expressed at a relatively low level in the TAXOL/cisplatin resistant clinical samples compared to the TAXOL/cisplatin sensitive clinical samples (Table 11B). Thus, Table 11A presents resistance genes and Table 11B presents sensitivity genes.

Based on these studies, various embodiments of the present invention are directed to uses of the specific identified genes whose expression is correlated with sensitivity or resistance to treatment with a therapeutic agent. In particular, the present invention provides: 1) methods for determining whether a particular therapeutic agent will be effective in stopping or slowing tumor progression; 2) methods for monitoring the effectiveness of therapeutic agents used for the treatment of cancer; 3) methods for developing new therapeutic agents for the treatment of cancer; and 4) methods for identifying combinations of therapeutic agents for the treatment of cancer.

In the present invention, two general classes of genes are identified: 1) genes that are expressed in cancer cell lines that are resistant to a given therapeutic agent and whose expression correlates with resistance to that therapeutic agent (“resistance genes”); and 2) genes that are expressed in cancer cell lines that are sensitive to a given agent and whose expression correlates with sensitivity to that therapeutic agent (“sensitivity genes”). Genes whose expression correlates with sensitivity to an agent are listed in Table 1 (positive scores), Table 2a (positive scores), Tables 7A-7D, Table 8A, Tables 9A-9D, Table 10A and Table 11A; and genes whose expression correlates with resistance to the agent are listed in Table 1 (negative scores), Table 2b (negative scores), Table 8B, Table 10B, and Table 11B.

By examining the expression of one or more of the identified sensitivity or resistance genes in a sample of cancer cells, it is possible to determine which therapeutic agent or combination of agents will be most likely to reduce the growth rate of the cancer and can further be used in selecting appropriate treatment agents.

By examining the expression of one or more of the identified resistance genes in a sample of cancer cells, it is possible to determine which therapeutic agent or combination of agents will be the least likely to reduce the growth rate of the cancer. By examining the expression of one or more of the identified resistance genes, it is possible to eliminate inappropriate therapeutic agents. By examining the expression of one or more sensitivity genes or resistance genes when cancer cells or a cancer cell line is exposed to a potential anti-cancer agent, it is possible to identify new anti-cancer agents. Lastly, by examining the expression of one or more of the identified sensitivity or resistance genes in a sample of cancer cells taken from a patient during the course of therapeutic treatment, it is possible to determine whether the therapeutic treatment is continuing to be effective or whether the cancer has become resistant (refractory) to the therapeutic treatment. Importantly, these determinations can be made on a patient by patient basis or on an agent by agent (or combination of agents) basis. Thus, one can determine whether or not a particular therapeutic treatment is likely to benefit a particular patient or group/class of patients, or whether a particular treatment should be continued.

The present invention further provides previously unknown or unrecognized targets for the development of anti-cancer agents, such as chemotherapeutic compounds. Both the identified sensitivity genes and the identified resistance genes of the present invention can be used as targets in developing treatments (either single agent or multiple agent) for cancer, particularly for those cancers which display resistance that is mediated by the expression of one or more of the resistance genes identified herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. in the case of conflict, the present specification, including definitions, will control. In addition, the materials, methods, and examples are illustrative only and are not intended to be limiting.

Other features and advantages of the invention will be apparent from the detailed description and from the claims. Although materials and methods similar or equivalent to those described herein can be used in the practice or testing of the invention, the preferred materials and methods are described below.

### DETAILED DESCRIPTION OF THE INVENTION

### General Description

The present invention is based, in part, on the identification of genes whose expression is correlated with sensitivity to or resistance to treatment with a therapeutic agent. In the present invention, two general classes of genes are identified: 1) genes that are expressed in cancer cell lines that are resistant to a given therapeutic agent and whose expression correlates with resistance to that agent; and 2) genes that are expressed in cancer cell lines that are sensitive to a given therapeutic agent and whose expression correlates with sensitivity to that therapeutic agent. Genes whose expression correlates with sensitivity to the agent are listed in Table 1 (positive scores), Table 2a (positive scores), Table 8B, Table 10B, and Table 11B; while genes whose expression correlates with resistance to the agent are listed in Table 1 (negative scores), Table 2b (negative scores), Tables 7A-7D, Table 8A, Tables 9A-9D, Table 10A and Table 11B.

Based on these identifications, the present invention provides: 1) methods for determining whether a therapeutic agent (or combination of agents) will or will not be effective in stopping or slowing tumor growth; 2) methods for monitoring the effectiveness of a therapeutic agent (or combination of agents) used for the treatment of cancer; 3) methods for identifying new therapeutic agents for the treatment of cancer; 4) methods for identifying combinations of therapeutic agents for use in treating cancer; and 5) methods for identifying specific therapeutic agents and combinations of therapeutic agents that are effective for the treatment of cancer in specific patients.

### Specific Embodiments

### Identification Of Sensitivity And Resistance Genes

Described below in the Examples is the identification of two classes of genes: 1) genes that are expressed in cancer cell lines that are resistant to a given therapeutic agent and whose expression correlates with resistance to that therapeutic agent; and 2) genes that are expressed in cancer cell lines that are sensitive to a given therapeutic agent and whose expression correlates with sensitivity to that therapeutic agent.

The Examples provided below concern the identification of genes that are expressed in cancer cell lines that are either sensitive or resistant to defined chemotherapeutic agents summarized in Tables 1, 2a, 2b, 7A-7D, 8A, 8B, 9A-9D, 10A, 10B, 11A, and 11B. As used herein, cancer cells are said to be sensitive to an agent if, at a therapeutic concentration, the agent can inhibit more than 50% of the growth of the cancer cells. As used herein, cancer cells are said to be resistant to an agent if, at a therapeutic concentration, the agent cannot inhibit more than 50% of the growth of the cancer cells.

Accordingly, one or more of the sensitivity genes that are expressed by cancer cell lines that are sensitive to treatment with an agent can be used as markers (or surrogate markers) to identify cancer cells that can be successfully treated by that agent. In addition, these genes can be used as markers to identify cancers that have become or at risk for becoming refractory to treatment with the agent.

A loss of expression of one or more of the sensitivity genes can be used as an indication that the cancer is or is at risk at becoming refractory to treatment. One or more of the resistance genes that are expressed by cancer cell lines resistant to treatment with an agent can be used as markers (or surrogate markers) to identify cancer cells that cannot be successfully treated by that agent. In addition, these genes can be used as markers (or surrogate markers) to identify cancers that have become or are at risk for becoming refractory to treatment with the agent.

### Determining Sensitivity or Resistance To An Agent

The expression level of the identified sensitivity and resistance genes, or the proteins encoded by the identified sensitivity and resistance genes, may be used to: 1) determine if a cancer can be treated by an agent or combination of agents; 2) determine if a cancer is responding to treatment with an agent or combination of agents; 3) select an appropriate agent or combination of agents for treating a cancer; 4) monitor the effectiveness of an ongoing treatment; and 5) identify new cancer treatments (either single agent or combination of agents). In particular, the identified sensitivity and resistance genes may be utilized as markers (surrogate and/or direct) to determine appropriate therapy, to monitor clinical therapy and human trials of a drug being tested for efficacy, and to develop new agents and therapeutic combinations.

Accordingly, the present invention provides methods for determining whether an agent, e.g., a chemotherapeutic agent, can be used to reduce the growth rate of cancer cells comprising the steps of:

a) obtaining a sample of cancer cells;

b) determining the level of expression in the cancer cells of one or more genes selected from the group consisting of the sensitivity genes (Table 1 (positive scores), Table 2a (positive scores), Table 8B, Table 10B, and Table 11B) and the resistance genes (Table 1 (negative scores), Table 2b (negative scores), Tables 7A-7D, Table 8A, Tables 9A-9D, Table 10A, and Table 11A); and

c) identifying that an agent can be used to treat the cancer when one or more of the sensitivity genes is expressed and/or when one or more of the resistance genes is not expressed.

Alternatively, in step (c), an agent can be identified as not being appropriate to use to treat the cancer when one or more of the sensitivity genes is not expressed and/or when one or more of the resistance genes is expressed.

As used herein, an agent is said to reduce the rate of growth of cancer cells when the agent can reduce at least 50%, preferably at least 75%, most preferably at least 95% of the growth of the cancer cells.

Such inhibition can further include a reduction in survivability and an increase in the rate of death of the cancer cells.

The amount of agent used for this determination will vary based on the agent selected. Typically, the amount will be a predefined therapeutic amount.

As used herein, the term “agent” is defined broadly as anything that cancer cells may be exposed to in a therapeutic protocol. In the context of the present invention, such agents include, but are not limited to, chemotherapeutic agents, such as anti-metabolic agents, e.g., Ara AC, 5-FU and methotrexate, antimitotic agents, e.g., TAXOL, inblastine and vincristine, alkylating agents, e.g., melphanlan, BCNU and nitrogen mustard, Topoisomerase II inhibitors, e.g., VW-26, topotecan and Bleomycin, strand-breaking agents, e.g., doxorubicin and DHAD, cross-linking agents, e.g., cisplatin and CBDCA, radiation and ultraviolet light.

Further to the above, the language “chemotherapeutic agent” is intended to include chemical reagents which inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases. The chemotherapeutic agents generally employed in chemotherapy treatments are listed below in Table A.

TABLE A

| | | NONPROPRIETARY |
|---|---|---|
| | | NAMES |
| CLASS | TYPE OF AGENT | (OTHER NAMES) |
| Alkylating | Nitrogen Mustards | Mechlorethamine (HN₂) |
| | | Cyclophosphamide |
| | | Ifosfamide |
| | | Melphalan (L-sarcolysin) |
| | | Chlorambucil |
| | Ethylenimines | Hexamethylmelamine |
| | And Methylmelamines | Thiotepn |
| | Alkyl Sulfonates | Busulfan |
| | Nitrosoureas | Carmustine (BCNU) |
| | | Lomustine (CCNU) |
| | | Semustine (methyl-CCNU) |
| | | Streptozocin |
| | | (streptozotocin) |
| | Triazenes | Decarbazine (DTIC; |
| | | dimethyltriazenoimi- |
| | | dazolecarboxamide) |
| | Alkylator | cis-diamminedichloroplatinum |
| | | II (CDDP) |
| Antimetabolites | Folic Acid | Methotrexate |
| | Analogs | (amethopterin) |
| | Pyrimidine | Fluorouracil |
| | Analogs | (′5-fluorouracil; 5-FU) |
| | | Floxuridine (fluorode- |
| | | oxyuridine; FUdR) |
| | | Cytarabine (cytosine |
| | | arabinoside) |
| | Purine Analogs | Mercaptopuine |
| | and Related | (6-mercaptopurine; |
| | Inhibitors | 6-MP) |
| | | Thioguanine |
| | | (6-thioguanine; TG) |
| | | Pentostatin |
| | | (2′-deoxycoformycin) |
| Natural | Vinca Alkaloids | Vinblastin (VLB) |
| Products | | Vincristine |
| | Topoisomerase | Etoposide |
| | Inhibitors | Teniposide |
| | | Camptothecin |
| | | Topotecan |
| | | 9-amino-campotothecin |
| | | CPT-11 |
| | Antibiotics | Dactinomycin |
| | | (actinomycin D) |
| | | Adriamycin |
| | | Daunorubicin |
| | | (daunomycin; |
| | | rubindomycin) |
| | | Doxorubicin |
| | | Bleomycin |
| | | Plicamycin |
| | | (mithramycin) |
| | | Mitomycin (mitomycin C) |
| | | Taxol |
| | | Taxotere |
| | Enzymes | L-Asparaginase |
| | Biological | Interfon alfa |
| | Response | interleukin 2 |
| | Modifiers | |
| Miscellaneous | Platinum | cis-diamminedichloroplatinum |
| Agents | Coordination | II (CDDP) |
| | Complexes | Carboplatin |
| | Anthracendione | Mitoxantrone |
| | Substituted Urea | Hydroxyurea |
| | Methyl Hydraxzine | Procarbazine |
| | Derivative | (N-methylhydrazine, |
| | | (MIH) |
| | Adrenocortical | Mitotane (o,p′-DDD) |
| | Suppressant | Aminoglutethimide |
| Hormones and | Adrenocorticosteroids | Prednisone |
| Antagonists | Progestins | Hydroxyprogesterone |
| | | caproate |
| | | Medroxyprogesterone |
| | | acetate |
| | | Megestrol acetate |
| | Estrogens | Diethylstilbestrol |
| | | Ethinyl estradiol |
| | Antiestrogen | Tamoxifen |
| | Androgens | Testosterone propionate |
| | | Fluoxymesterone |
| | Antiandrogen | Flutamide |
| | Gonadotropin-releasing | Leuprolide |
| | Hormone analog | |

The agents tested in the present methods can be a single agent or a combination of agents. For example, the present methods can be used to determine whether a single chemotherapeutic agent, such as methotrexate, can be used to treat a cancer or whether a combination of two or more agents can be used. Preferred combinations will include agents that have different mechanisms of action, e.g., the use of an anti-mitotic agent in combination with an alkylating agent. For example, using the data provided in Table 1, to determine sensitivity/resistance to 5-FU, the sensitivity genes are selected from the group consisting of transcription factor btf3 and major gastrointestinal tumor-associated protein ga73 3-2 and the resistance genes are selected from the group consisting of fibrinogen alpha chain precursor, fibrinogen gamma-a chain precursor, complement c4 precursor, and fibrinogen beta chain precursor.

As used herein, cancer cells refer to cells that divide at an abnormal (increased) rate. Cancer cells include, but are not limited to, carcinomas, such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; leukemias and lymphomas such as granulocytic leukemia, monocytic leukemia, lymphocytic leukemia, malignant lymphoma, plasmocytoma, reticulum cell sarcoma, or Hodgkins disease; and tumors of the nervous system including glioma, meningoma, medulloblastoma, schwannoma or epidymoma.

The source of the cancer cells used in the present method will be based on how the method of the present invention is being used. For example, if the method is being used to determine whether a patient's cancer can be treated with an agent, or a combination of agents, then the preferred source of cancer cells will be cancer cells obtained from a cancer biopsy from the patient. Alternatively, a cancer cell line similar to the type of cancer being treated can be assayed. For example if breast cancer is being treated, then a breast cancer cell line can be used. If the method is being used to monitor the effectiveness of a therapeutic protocol, then a tissue sample from the patient being treated is the preferred source. If the method is being used to identify new therapeutic agents or combinations, any cancer cells, e.g., cells of a cancer cell line, can be used.

A skilled artisan can readily select and obtain the appropriate cancer cells that are used in the present method. For cancer cell lines, sources such as The National Cancer Institute, for the NCI-60 cells used in the examples, are preferred. For cancer cells obtained from a patient, standard biopsy methods, such as a needle biopsy, can be employed.

In the methods of the present invention, the level or amount of expression of one or more genes selected from the group consisting of the genes identified in the Tables, is determined. As used herein, the level or amount of expression refers to the absolute level of expression of an mRNA encoded by the gene or the absolute level of expression of the protein encoded by the gene (i.e., whether or not expression is or is not occurring in the cancer cells).

Generally, it is preferable to determine the expression of two or more of the identified sensitivity or resistance genes, more preferably, three or more of the identified sensitivity or resistance genes, most preferably all of the identified sensitivity and/or resistance genes. Thus, it is preferable to assess the expression of a panel of sensitivity and resistance genes.

As an alternative to making determinations based on the absolute expression level of selected genes, determinations may be based on the normalized expression levels. Expression levels are normalized by correcting the absolute expression level of a sensitivity or resistance gene by comparing its expression to the expression of a gene that is not a sensitivity or resistance gene, e.g., a housekeeping genes that is constitutively expressed. Suitable genes for normalization include housekeeping genes such as the actin gene. This normalization allows one to compare the expression level in one sample, e.g., a patient sample, to another sample, e.g., a non-cancer sample, or between samples from different sources.

Alternatively, the expression level can be provided as a relative expression level. To determine a relative expression level of a gene, the level of expression of the gene is determined for 10 or more samples, preferably 50 or more samples, prior to the determination of the expression level for the sample in question. The mean expression level of each of the genes assayed in the larger number of samples is determined and this is used as a baseline expression level for the gene(s) in question. The expression level of the gene determined for the test sample (absolute level of expression) is then divided by the mean expression value obtained for that gene. This provides a relative expression level and aids in identifying extreme cases of sensitivity or resistance.

Preferably, the samples used will be from similar tumors or from non-cancerous cells of the same tissue origin as the tumor in question. The choice of the cell source is dependent on the use of the relative expression level data. For example, using tumors of similar types for obtaining a mean expression score allows for the identification of extreme cases of sensitivity or resistance. Using expression found in normal tissues as a mean expression score aids in validating whether the sensitivity/resistance gene assayed is tumor specific (versus normal cells). Such a later use is particularly important in identifying whether a sensitivity or resistance gene can serve as a target gene. In addition, as more data is accumulated, the mean expression value can be revised, providing improved relative expression values based on accumulated data.

In addition to detecting the level of expression of sensitivity, resistance, and normalization genes, in some instances it will also be import to monitor the level of expression of genes that indicate cell viability. The expression of such genes can be used as markers of the specificity of any particular agent, or combination, tested.

The expression level can be measured in a number of ways, including, but not limited to: measuring the mRNA encoded by the selected genes; measuring the amount of protein encoded by the selected genes; and measuring the activity of the protein encoded by the selected genes.

The mRNA level can be determine in in situ and in in vitro formats using methods known in the art. Many of such methods use isolated RNA. For in vitro methods, any RNA isolation technique that does not select against the isolation of mRNA can be utilized for the purification of RNA from the cancer cells (see, e.g., Ausubel et al., eds., 1987-1997, Current Protocols in Molecular Biology, John Wiley & Sons, Inc., New York). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic methods for the detection of mRNA levels involves contacting the isolated mRNA with a nucleic acid molecule (probe) that can hybridize to the mRNA encoded by the gene being detected. In one format, the mRNA is immobilized on a solid surface and contacted with the probes, for example by running the isolated mRNA on an agarose gel and transferring the mRNA from the gel to a membrane, such a nitrocellulose. In an alternative format, the probes are immobilized on a solid surface and the mRNA is contact with the probes, for example in an Affymetrix gene array. A skilled artisan can readily adapt known mRNA detection methods for use in detecting the level of mRNA encoded by one or more of the sensitivity genes or resistance genes of the present invention.

An alternative method for determining the level of mRNA in a sample that is encoded by one of the sensitivity or resistance genes of the present invention involves the process of nucleic acid amplification, e.g., by rtPCR (the experimental embodiment set forth in Mullis, 1987, U.S. Pat. No. 4,683,202), ligase chain reaction (Barany, 1991, Proc. Natl. Acad. Sci. USA 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988,Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

For in situ methods, mRNA does not need to be isolated from the cancer cells prior to detection. In such methods, a cell or tissue sample is prepared/processed using known histological methods. The sample is then immobilized on a support, typically a glass slide, and then contacted with a probe that can hybridize to mRNA that encodes the sensitivity or resistance gene being analyzed. Hybridization with the probe indicates that the gene in question is being expressed.

In analyzing mRNA that encodes a particular sensitivity or resistance gene, either a hybridization probe or a set of amplification primers are used. As used herein, a probe is defined as a nucleic acid molecule of at least 10 nucleotides, preferably at least 20 nucleotides, most preferably at least 30 nucleotides, that is complementary to the coding sequence of a resistance or sensitivity gene. As such, a probe will hybridize, preferably selectively hybridize, to the resistance or sensitivity gene that is obtained from. A skilled artisan can readily determine appropriate probes (both nucleotide sequence and length) for detecting the sensitivity and resistance genes of the present invention using art known methods and the nucleotide sequence of the sensitivity and resistance genes of the present invention.

As used herein, amplification primers are defined as being a pair of nucleic acid molecules that can anneal to 5′ or 3′ regions of a gene (plus and minus strands respectively or visa-versa) and contain a short region in between. In general, amplification primers are from about 10 to 30 nucleotides in length and flank a region from about 50 to 200 nucleotides in length. Amplification primers can be used to produce a nucleic acid molecule comprising the nucleotide sequence flanked by the primers. A skilled artisan can readily determine appropriate primers (both nucleotide sequence and length) for amplifying and detecting the sensitivity and resistance genes of the present invention using art known methods and the nucleotide sequence of the sensitivity and resistance genes of the present invention.

A variety of methods can be used to determine the level of protein encoded by one or more of the sensitivity or resistance genes of the present invention. In general, these method involve the use of a compound that selectively binds to the protein, for example an antibody.

Proteins from cancer cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Example of such formats include, but are not limited to enzyme immunoassay (EIA), radioimmunoassay (RIA), Western blot analysis and enzyme linked immunoabsorbant assay (ELISA). A skilled artisan can readily adapt know protein/antibody detection methods for use in determining whether cancer cells expresses a protein encoded by one or more of the sensitivity or resistance genes of the present invention.

In one format, antibodies, or antibody fragments, can be used in methods such as Western blots or immunofluorescence techniques to detect the expressed proteins. In such uses, it is generally preferable to immobilize either the antibody or protein on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from cancer cells can be run on a polyacrylamide gel electrophoresis and immobilized onto a solid phase support such as nitrocellulose. The support can then be washed with suitable buffers followed by treatment with the detectably labeled sensitivity or resistance gene product specific antibody. The solid phase support can then be washed with the buffer a second time to remove unbound antibody. The amount of bound label on the solid support can then be detected by conventional means.

Another embodiment of the present invention includes a step of detecting whether an agent stimulates the expression of one or more of the sensitivity or resistance genes of the present invention. Although some of the present sensitivity and resistance genes were identified as being expressed in non-treated cancer cells, treatment with an agent may, or may not, alter expression. Alterations in the expression level of the sensitivity and resistance genes of the present invention can provide a further indication as to whether an agent will or will not be effective at reducing the growth rate of the cancer cells. In such a use, the present invention provides methods for determining whether an agent, e.g., a chemotherapeutic agent, can be used to reduce the growth rate of cancer cells comprising the. steps of:

a) obtaining a sample of cancer cells;

b) exposing the sample of cancer cells to one or more test agents;

c) determining the level of expression in the cancer cells of one or more genes selected from the group consisting of the genes identified in the Tables in the sample exposed to the agent and in a sample of cancer cells that is not exposed to the agent; and

d) identifying that an agent can be used to treat the cancer when the expression of one or more of the sensitivity genes is increased in the presence of said agent and/or when the expression of one or more of the resistance genes is not increased in the presence of said agent.

Alternatively, in step (d), an agent can be identified as not being appropriate to use to treat the cancer when the expression of one or more of the sensitivity genes is not increased in the presence of said agent and/or when the expression of one or more of the resistance genes is increased in the presence of said agent.

This embodiment of the methods of the present invention involves the step of exposing the cancer cells to an agent. The method used for exposing the cancer cells to the agent will be based primarily on the source and nature of the cancer cells and the agent being tested. The contacting can be performed in vitro or in vivo, in a patient being treated/evaluated or in animal model of a cancer. For cancer cells and cell lines and chemical compounds, exposing the cancer cells involves contacting the cancer cells with the compound, such as in tissue culture media. A skilled artisan can readily adapt an appropriate procedure for contacting cancer cells with any particular agent or combination of agents.

### Monitoring the Effectiveness of a Chemotherapeutic Agent

As discussed above, the identified sensitivity and resistance genes can also be used as markers to assess whether a tumor has become refractory to an ongoing treatment (e.g., a chemotherapeutic treatment). When a tumor is no longer responding to a treatment the expression profile of the tumor cells will change: the level of expression of one or more of the sensitivity genes will be reduced and the level of expression of one or more of the resistance genes will increase.

In such a use, the invention provides methods for determining whether an anti-cancer treatment should be continued in a cancer patient, comprising the steps of:

a) obtaining two or more samples of cancer cells from a patient undergoing anti-cancer therapy;

b) determining the level of expression of one or more genes selected from the group consisting of the sensitivity genes and the resistance genes in the sample exposed to the agent and in a sample of cancer cells that is not exposed to the agent; and

c) discontinuing treatment when the expression of one or more sensitivity genes decreases or when the expression of one or more resistance genes increases.

As used here, a patient refers to any subject undergoing treatment for cancer. The preferred subject will be a human patient undergoing chemotherapy treatment.

This embodiment of the present invention relies on comparing two or more samples obtained from a patient undergoing anti-cancer treatment. In general, it is preferable to obtain a first sample from the patient prior to beginning therapy and one or more samples during treatment. In such a use, a baseline of expression prior to therapy is determined and then changes in the baseline state of expression is monitored during the course of therapy. Alternatively, two or more successive sample obtained during treatment can be used without the need of a pre-treatment baseline sample. In such a use, the first sample obtained from the subject is used as a baseline for determining whether the expression of a particular gene is increasing or decreasing.

In general, when monitoring the effectiveness of a therapeutic treatment, two or more samples from the patient are examined. Preferably, three or more successively obtained samples are used, including at least one pretreatment sample.

### Kits Containing Reagents for Conducting the Methods of the Present Invention

The present invention further provides kits comprising compartmentalized containers comprising reagents for detecting one or more, preferably two or more, of the sensitivity and resistance genes of the present invention. As used herein a kit is defined as a pre-packaged set of containers into which reagents are placed. The reagents included in the kit comprise probes/primers and/or antibodies for use in detecting sensitivity and resistance gene expression. In addition, the kits of the present invention may preferably contain instructions which describe a suitable detection assay. Such kits can be conveniently used, e.g., in clinical settings, to diagnose patients exhibiting symptoms of cancer.

### Further Characterization of the Sensitivity and Resistance Genes

Sensitivity and resistance genes can be further characterized by using techniques known to those skilled in the art to yield more information regarding potential targets for the therapeutic treatment of cancer and for identifying other sensitivity and resistance genes. For example, characterization of the identified sensitivity and resistance genes can yield information regarding the biological function of the identified genes.

Specifically, any of the sensitivity and resistance genes whose further characterization indicates that a modulation of the gene's expression or a modulation of the gene product's activity can reduce symptoms of cancer are designated “target genes.” As used herein, a target gene is a gene (or gene product) that when modulated, can provide therapeutic treatment of the cancer. As such target genes and target gene products can be used to identify therapeutics agents. Sensitivity and resistance genes whose further characterization indicates that it does not influence growth or viability of cancer cells, but whose expression pattern contributes to a gene expression pattern correlative of, for example, the effectiveness of a drug is designated a “sensitivity gene” or “resistance gene” and cannot serve as a target gene. Such genes can be used as diagnostic markers and as markers for assessing the effectiveness or potential effectiveness of a therapeutic agent.

A variety of techniques can be utilized to further characterize the identified sensitivity and resistance genes herein identified. First, the nucleotide sequence of the identified genes, obtained by standard techniques well known to those of skill in the art, can be used to further characterize such genes. For example, the sequence of the identified genes can reveal homologies to one or more known sequence motifs that can yield information regarding the biological function of the identified gene product.

Second, an analysis of the tissue and/or cell type distribution of the mRNA produced by the identified genes can be conducted, utilizing standard techniques well known to those of skill in the art. Such techniques can include, for example, Northern analyses, RT-coupled PCR and RNase protection techniques. Such analyses can be used to determine whether the identified genes are expressed in tissues expected to contribute to cancer, whether the genes are highly regulated in tissues that can be expected to contribute to cancer, and whether cells within a given tissue express the identified gene. Such an analysis can provide information regarding the biological function of an identified gene in instances wherein only a subset of the cells within the tissue is thought to be relevant to cancer.

Third, the sequences of the identified genes can be used, utilizing standard techniques, to place the genes onto genetic maps, e.g., mouse (Copeland and Jenkins 1991, Trends in Genetics 7:113-118) and human genetic maps (Cohen et al., 1993, Nature 366:698-701). Such mapping information can yield information regarding the genes' importance to human disease by, for example, identifying genes that map within a genetic region to which predisposition to cancer also maps.

Fourth, the biological function of the identified genes can be more directly assessed by utilizing relevant in vivo and in vitro systems. In vivo systems can include, but are not limited to, animal systems that naturally exhibit symptoms of cancer or ones that have been engineered to exhibit such symptoms.

The role of identified gene products can be determined by transfecting cDNAs encoding these gene products into appropriate cell lines, such as, for example, cancer cells line and analyzing the effect of the gene product on cell growth.

In further characterizing the biological function of the identified genes, the expression of these genes can be modulated within the in vivo and/or in vitro systems, i.e., either over-expressed or under-expressed, and the subsequent effect on the system then assayed. Alternatively, the activity of the product of the identified gene can be modulated by either increasing or decreasing the level of activity in the in vivo and/or in vitro system of interest, and assessing the effect of such modulation.

The information obtained through such characterizations can suggest relevant methods for the treatment of cancer. For example, treatment can include a modulation of gene expression and/or gene product activity. Characterization procedures such as those described herein can indicate where such modulation should involve an increase or a decrease in the expression or activity of the gene or gene product of interest.

### Identification of Compounds that Interact with a Target Gene Product

The following assays are designed to identify compounds that bind to target gene products, compounds that bind to other cellular proteins that interact with a target gene product, and compounds that interfere with the interaction of the target gene product with other cellular proteins.

Such compounds can include, but are not limited to, other cellular proteins, natural products and small chemical molecules. Specifically, such compounds can include, but are not limited to, peptides, soluble peptides, Ig-tailed fusion peptides, extracellular portions of target gene product transmembrane receptors, members of random peptide libraries (see, e.g., Lam et al., 1991, Nature 354:82-84; Houghton et al., 1991, Nature 354:84-86) made of D-and/or L-configuration amino acids, phosphopeptides (including, but not limited to, members of random or partially degenerate phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Compounds identified via assays such as those described herein can be useful, for example, in elaborating the biological function of the target gene product, and for ameliorating symptoms of cancer. For example, for sensitivity genes, compounds that interact with the gene product of the sensitivity gene can be used to treat the cancer. For resistance genes, compounds that decrease the level of expression of the resistance gene or the activity of the encoded protein, can serve as a therapeutic agent.

### Screening Assays for Compounds and Cellular Proteins that Bind to a Target Gene Product

In vitro systems can be designed to identify compounds capable of binding the target gene products of the invention. Compounds thus identified can be used to modulate the activity of target gene products in a therapeutic protocol, to elaborate the biological function of the target gene product, or to identify compounds that disrupt normal target gene interactions. The preferred targets genes/products used in this embodiment are the sensitivity genes and resistance genes of the present invention.

The principle of the assays used to identify compounds that bind to the target gene product involves preparing a reaction mixture of the target gene protein and the test compound under conditions and for a time sufficient to allow the two components to interact and bind, thus forming a complex that can be removed and/or detected in the reaction mixture. These assays can be conducted in a variety of ways. For example, one method to conduct such an assay would involve anchoring target gene product or the test substance onto a solid phase and detecting target gene product/test compound complexes anchored on the solid phase at the end of the reaction. In one embodiment of such a method, the target gene product can be anchored onto a solid surface, and the test compound, which is not anchored, can be labeled, either directly or indirectly.

In practice, microtiter plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any specific complexes formed will remain immobilized on the solid surface. The detection of complexes anchored on the solid surface can be accomplished in a number of ways. Where the previously immobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for target gene or the test compound to anchor any complexes formed in solution, and a labeled antibody specific for the other component of the possible complex to detect anchored complexes.

Any method suitable for detecting protein-protein interactions can be employed for identifying novel target product-cellular or extracellular protein interactions. In such a case, the target gene serves as the known “bait” gene.

### Assays for Compounds that Interfere with the Binding of a Target Gene Product to a Second Cellular Protein

The target gene products of the invention can, in vivo, interact with one or more cellular or extracellular macromolecules, such as proteins. For the purposes of this discussion, such cellular and extracellular macromolecules are referred to as “binding partners.” Compounds that disrupt such interactions can be useful in regulating the activity of the target gene product. Such compounds can include, but are not limited to, molecules such as antibodies, peptides, and small molecules. The preferred target genes/products for use in this embodiment are the resistance genes herein identified.

The basic principle of the assay systems used to identify compounds that interfere with the interaction between the target gene product and its cellular or extracellular binding partner or partners involve preparing a reaction mixture containing the target gene product and the binding partner under conditions and for a time sufficient to allow the target gene product and its binding partner to interact and bind, thus forming a complex. In order to test an agent for inhibitory activity, the reaction mixture is prepared in the presence and absence of the test compound. The test compound can initially be included in the reaction mixture, or can be added at a time subsequent to the addition of target gene and its cellular or extracellular binding partner. Control reaction mixtures are incubated without the test compound or with a placebo. The formation of any complexes between the target gene product and the cellular or extracellular binding partner is then detected. The formation of a complex in the control reaction, but not in the reaction mixture containing the test compound, indicates that the compound interferes with the interaction of the target gene product and its binding partner. Additionally, complex formation within reaction mixtures containing the test compound and normal target gene product can also be compared to complex formation within reaction mixtures containing the test compound and mutant target gene product. This comparison can be important in those cases wherein it is desirable to identify compounds that disrupt interactions of mutant but not normal target gene products.

The assay for compounds that interfere with the interaction of the target gene products and binding partners can be conducted in a heterogeneous or homogeneous format. Heterogeneous assays involve anchoring either the target gene product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between a selected target gene product and its binding partners, e.g., by competition, can be identified by conducting the reaction in the presence of the test substance; i.e., by adding the test substance to the reaction mixture prior to or simultaneously with the target gene product and its binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the target gene product or its binding partner is anchored onto a solid surface, while the non-anchored species is labeled, either directly or indirectly. In practice, microtitre plates are conveniently utilized. The anchored species can be immobilized by non-covalent or covalent attachment. Non-covalent attachment can be accomplished simply by coating the solid surface with a solution of the target gene product or binding partner and drying. Alternatively, an immobilized antibody specific for the species to be anchored can be used to anchor the species to the solid surface. The surfaces can be prepared in advance and stored.

In order to conduct the assay, the partner of the immobilized species is exposed to the coated surface in the presence and absence of the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes immobilized on the surface; e.g., using a labeled antibody specific for the initially non-immobilized species (the antibody, in turn, can be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound. In this format, the reaction products separated from unreacted components and any complexes detected, e.g., using an immobilized antibody specific for one of the binding components to anchor any complexes formed in solution and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a preformed complex of the target gene product and binding partner is prepared such that either the target gene product or its binding partner is labeled and the signal generated by the label is quenched by complex formation (see, e.g., U.S. Pat. No. 4,109,496 that utilizes this approach for immunoassays). The addition of a test substance that competes with and displaces one of the species from the preformed complex will result in the generation of a signal above background. In this way, test substances that disrupt target gene product-cellular or extracellular binding partner interaction can be identified.

### Assays Based On Target Gene Product Activity

The present invention further provides methods for identifying new anti-cancer agents or combinations that are based on identifying agents that modulate the activity of one or more of the gene products encoded by one or more of the sensitivity or resistance genes of the present invention. Specifically, the activity of the proteins encoded by the resistance genes of the present invention can be used as a basis for identifying agents for overcoming agent resistance. Specifically, by blocking the activity of one or more of the resistance proteins, cancer cells will become sensitive to treatment with an agent that the unmodified cancer cells were resistant to.

The choice of assay format will be based primarily on the nature and type of sensitivity or resistance protein being assayed. A skilled artisan can readily adapt protein activity assays for use in the present invention with the genes identified herein. For example, DNA ligase activity can be measured using art known methods.

### Treatment of Cancer by Modulation of Sensitivity and Resistance Genes or Gene Products

Cancer can be treated by modulating the expression of a target gene or the activity of a target gene product. The modulation can be of a positive or negative nature, depending on the specific situation involved, but in either case, the modulatory event results in amelioration of cancer symptoms.

“Negative modulation,” refers to a reduction in the level and/or activity of target gene product relative to the level and/or activity of the target gene product in the absence of the modulatory treatment.

“Positive modulation,” refers to an increase in the level and/or activity of target gene product relative to the level and/or activity of target gene product in the absence of modulatory treatment.

It is possible that cancer can be caused, at least in part, by an abnormal level of gene product, or by the presence of a gene product exhibiting abnormal activity. As such, the reduction in the level and/or activity of such gene products would bring about the amelioration of cancer symptoms.

Alternatively, it is possible that cancer can be brought about, at least in part, by the absence or reduction of the level of gene expression, or a reduction in the level of a gene product's activity. As such, an increase in the level of gene expression and/or the activity of such gene products would bring about the amelioration of cancer symptoms.

### Negative Modulatory Techniques

As discussed, above, successful treatment of cancer can be brought about by techniques that serve to inhibit the expression or activity of one or more target gene products.

For example, a compound e.g., an agent identified using an assays described above, that proves to exhibit negative modulatory activity, can be used in accordance with the invention to prevent and/or ameliorate symptoms of cancer. Such molecules can include, but are not limited to peptides, phosphopeptides, small organic or inorganic molecules, or antibodies (including, for example, polyclonal, monoclonal, humanized, anti-idiotypic, chimeric or single chain antibodies, and FAb, F(ab′)₂ and FAb expression library fragments, and epitope-binding fragments thereof).

Further, antisense and ribozyme molecules that inhibit expression of the target gene can also be used in accordance with the invention to reduce the level of target gene expression, thus effectively reducing the level of target gene activity. Still further, triple helix molecules can be utilized in reducing the level of target gene activity.

Among the compounds that can exhibit the ability to prevent and/or ameliorate symptoms of cancer are antisense, ribozyme, and triple helix molecules. Such molecules can be designed to reduce or inhibit either wild type, or if appropriate, mutant target gene activity. Techniques for the production and use of such molecules are well known to those of skill in the art.

Anti-sense RNA and DNA molecules act to directly block the translation of mRNA by hybridizing to targeted mRNA and preventing protein translation. With respect to antisense DNA, oligodeoxyribonucleotides derived from the translation initiation site, e.g., between the −10 and +10 regions of the target gene nucleotide sequence of interest, are preferred.

Ribozymes are enzymatic RNA molecules capable of catalyzing the specific cleavage of RNA. (For a review, see, for example, Rossi, 1994, Current Biology 4:469-471.) The mechanism of ribozyme action involves sequence specific hybridization of the ribozyme molecule to complementary target RNA, followed by a endonucleolytic cleavage. The composition of ribozyme molecules must include one or more sequences complementary to the target gene mRNA and must include the well-known catalytic sequence responsible for mRNA cleavage. For this sequence, see U.S. Pat. No. 5,093,246, which is incorporated by reference herein in its entirety. As such within the scope of the invention are engineered hammerhead motif ribozyme molecules that specifically and efficiently catalyze endonucleolytic cleavage of RNA sequences encoding target gene proteins.

Specific ribozyme cleavage sites within any potential RNA target are initially identified by scanning the molecule of interest for ribozyme cleavage sites that include the following sequences, GUA, GUU, and GUC. Once identified, short RNA sequences of between 15 and 20 ribonucleotides corresponding to the region of the target gene containing the cleavage site can be evaluated for predicted structural features, such as secondary structure, that can render the oligonucleotide sequence unsuitable. The suitability of candidate sequences can also be evaluated by testing their accessibility to hybridization with complementary oligonucleotides, using ribonuclease protection assays.

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, that generally require sizeable stretches of either purines or pyrimidines to be present on one strand of a duplex. Nucleotide sequences can be pyrimidine-based, that will result in TAT and CGC⁺ triplets across the three associated strands of the resulting triple helix. The pyrimidine-rich molecules provide base complementarily to a purine-rich region of a single strand of the duplex in a parallel orientation to that strand. In addition, nucleic acid molecules can be chosen that are purine-rich, e.g., contain a stretch of G residues. These molecules will form a triple helix with a DNA duplex that is rich in GC pairs, in that the majority of the purine residues are located on a single strand of the targeted duplex, resulting in GGC triplets across the three strands in the triplex.

Alternatively, the potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to reduce or inhibit mutant gene expression, it is possible that the technique utilized can also efficiently reduce or inhibit the transcription (triple helix) and/or translation (antisense, ribozyme) of mRNA produced by normal target gene alleles such that the concentration of normal target gene product present can be lower than is necessary for a normal phenotype. In such cases, to ensure that substantially normal levels of target gene activity are maintained, nucleic acid molecules that encode and express target gene polypeptides exhibiting normal target gene activity can be introduced into cells via gene therapy methods. Alternatively, in instances in that the target gene encodes an extracellular protein, it can be preferable to co-administer normal target gene protein into the cell or tissue in order to maintain the requisite level of cellular or tissue target gene activity.

Anti-sense RNA and DNA, ribozyme and triple helix molecules of the invention can be prepared by any method known in the art for the synthesis of DNA and RNA molecules. These include techniques for chemically synthesizing oligodeoxyribonucleotides and oligoribonucleotides that are well known in the art such as, for example, solid phase phosphoramidite chemical synthesis. Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Various well-known modifications to the DNA molecules can be introduced as a means of increasing intracellular stability and half-life. Possible modifications include but are not limited to the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone.

Antibodies can be generated that are specific for target gene product and reduce target gene product activity. Such antibodies may, therefore, by administered in instances whereby negative modulatory techniques are appropriate for the treatment of cancer. Antibodies can be generated using standard techniques against the proteins themselves or against peptides corresponding to portions of the proteins. The antibodies include but are not limited to polyclonal, monoclonal, Fab fragments, single chain antibodies, chimeric antibodies, and the like.

In instances where the target gene protein to which the antibody is directed is intracellular and whole antibodies are used, internalizing antibodies are preferred. However, lipofectin or liposomes can be used to deliver the antibody or an antigen binding fragment thereof into cells. Where fragments of the antibody are used, the smallest inhibitory fragment that binds to the target protein in an effective manner is referred. For example, peptides having an amino acid sequence corresponding to the domain of the variable region of the antibody that binds to the target gene protein can be used. Such peptides can be synthesized chemically or produced via recombinant DNA technology using methods well known in the art (e.g., see Creighton, 1983, supra; and Sambrook et al., 1989, supra). Alternatively, single chain neutralizing antibodies that bind to intracellular target gene product epitopes can also be administered. Such single chain antibodies can be administered, for example, by expressing nucleotide sequences encoding single-chain antibodies within the target cell population by utilizing, for example, techniques such as those described in Marasco et al. (1993, Proc. Natl. Acad. Sci. USA 90:7889-7893).

### Therapeutic Treatment

The identified compounds that inhibit target gene expression, synthesis and/or activity can be administered to a patient at therapeutically effective doses to prevent, treat or ameliorate cancer. A therapeutically effective dose refers to that amount of the compound sufficient to result in amelioration of symptoms of cancer.

### Effective Dose

Toxicity and therapeutic efficacy of therapeutic compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in designing a dosage range for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

### Formulations And Use

Pharmaceutical compositions for use in accordance with the present invention can be formulated in conventional manner using one or more physiologically acceptable carriers or excipients.

Thus, the compounds and their physiologically acceptable salts and solvates can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray from pressurized packs or a nebulizer, using a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

### SPECIFIC EXAMPLES

At least some of the examples set forth below relate to sensitivity and resistance to TAXOL. TAXOL is a chemical compound within a family of taxane compounds which are art-recognized as being a family of related compounds. The language “taxane compound” is intended to include TAXOL, compounds which are structurally similar to TAXOL and/or analogs of TAXOL. The language “taxane compound” can also include “mimics”. “Mimics” is intended to include compounds which may not be structurally similar to TAXOL but mimic the therapeutic activity of TAXOL or structurally similar taxane compounds in vivo. The taxane compounds of this invention are those compounds which are useful for inhibiting tumor growth in subjects (patients). The term taxane compound also is intended to include pharmaceutically acceptable salts of the compounds. Taxane compounds have previously been described in U.S. Pat. Nos. 5,641,803, 5,665,671, 5,380,751, 5,728,687, 5,415,869, 5,407,683, 5,399,363, 5,424,073, 5,157,049, 5,773,464, 5,821,263, 5,840,929, 4,814,470, 5,438,072, 5,403,858, 4,960,790, 5,433,364, 4,942,184, 5,362,831, 5,705,503, and 5,278,324, all of which are expressly incorporated by reference.

The structure of TAXOL, shown below, offers many groups capable of being synthetically functionalized to alter the physical or pharmaceutical properties of TAXOL.

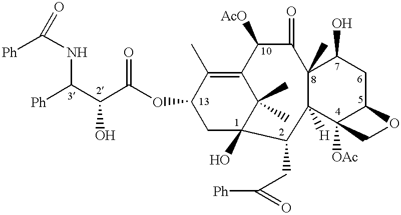

For example, a well known semi-synthetic analog of TAXOL, named Taxotere (docetaxel), has also been found to have good anti-tumor activity in animal models. Taxotere has t-butoxy amide at the 3′ position and a hydroxyl group at the C10 position (U.S. Pat. No. 5,840,929).

Other examples of TAXOL derivatives include those mentioned in U.S. Pat. No. 5,840,929 which are directed to derivatives of TAXOL having the formula:

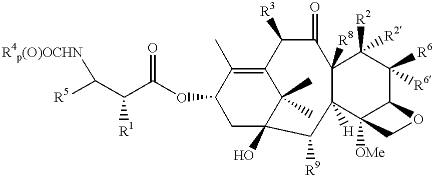

wherein R¹ is hydroxy, —OC(O)R^{x}, or —OC(O)OR^{x}; R² is hydrogen, hydroxy, —OC(O)R^{x}, or —OC(O)OR^{x}; R^{2′} is hydrogen, hydroxy, or fluoro; R^{6′} is hydrogen or hydroxy or R^{2′} and R^{6′} can together form an oxirane ring; R³ is hydrogen, C₁₋₆ alkyloxy, hydroxy, —OC(O)R^{x}, —OC(O)OR^{x}, —OCONR⁷R¹¹; R⁸ is methyl or R⁸ and R² together can form a cyclopropane ring; R⁶ is hydrogen or R⁶ and R² can together form a bond; R⁹ is hydroxy or —OC(O)R^{x}; R⁷ and R¹¹ are independently C₁₋₆ alkyl, hydrogen, aryl, or substituted aryl; R⁴ and R⁵ are independently C₁₋₆ alkyl, C₂₋₆ alkenyl, C₂₋₆ alkynyl, or —Z—R¹⁰; Z is a direct bond, C₁₋₆ alkyl, or C₂₋₆ alkenyl; R¹⁰ is is aryl, substituted aryl, C₃₋₆ cycloalkyl, C₂₋₆ alkenyl, C₁₋₆ alkyl, all can be optionally substituted with one to six same or different halogen atoms or hydroxy; R^{x} is a radical of the formula:

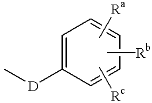

wherein D is a bond or C₁₋₆ alkyl; and R^{a}, R^{b} and R^{c} are independently hydrogen, amino, C₁₋₆ alkyl or C₁₋₆ alkoxy.

Further examples of R^{x} include methyl, hydroxymethyl, ethyl, n-propyl, isopropyl, n-butyl, isobutyl, chloromethyl, 2,2,2-trichloroethyl, cyclopropyl, cyclobutyl, cyclopentyl, cyclohexyl, ethenyl, 2-propenyl, phenyl, benzyl, bromophenyl, 4-aminophenyl, 4-methylaminophenyl, 4-methylphenyl, 4-methoxyphenyl and the like. Examples of R⁴ and R⁵ include 2-propenyl, isobutenyl, 3-furanyl (3-furyl), 3-thienyl, phenyl, naphthyl, 4-hydroxyphenyl, 4-methoxyphenyl, 4-fluorophenyl, 4-trifluoromethylphenyl, methyl, ethyl, n-propyl, isopropyl, n-butyl, isobutyl, t-butyl, ethenyl, 2-propenyl, 2-propynyl, benzyl, phenethyl, phenylethenyl, 3,4-dimethoxyphenyl, 2-furanyl (2-furyl), 2-thienyl, 2-(2-furanyl)ethenyl, 2-methylpropyl, cyclopropyl, cyclobutyl, cyclopentyl, cyclohexyl, cyclohexylmethyl, cyclohexylethyl and the like.

TAXOL derivatives can be readily made by following the well established paclitaxel chemistry. For example, C2, C6, C7, C10, and/or C8 position can be derivatized by essentially following the published procedure, into a compound in which R³, R8, R², R^{2′}, R⁹, R^{6′} and R⁶ have the meanings defined earlier. Subsequently, C4-acetyloxy group can be converted to the methoxy group by a sequence of steps. For example, for converting C2-benzoyloxy to other groups see, S. H. Chen et al, Bioorganic and Medicinal Chemistry Letters, Vol. 4, No. 3, pp 479-482 (1994); for modifying C10-acetyloxy see, J. Kant et al, Tetrahedron Letters, Vol. 35, No. 31, pp 5543-5546 (1994) and U.S. Pat. No. 5,294,637 issued Mar. 15, 1994; for making C10 and/or C7 unsubstituted (deoxy) derivatives see, European Patent Application 590 267A2 published Apr. 6, 1994 and PCT application WO 93/06093 published Apr. 1, 1993; for making 7β,8β-methano, 6,7-α,α-dihydroxy and 6,7-olefinic groups see, R. A. Johnson, Tetrahedron Letters, Vol. 35, No 43, pp 7893-7896 (1994), U.S. Pat. No. 5,254,580, issued Oct. 19, 1993, and European Patent Application 600 517A1 published Jun. 8, 1994; for making C7/C6 oxirane see, U.S. Pat. No. 5,395,850 issued Mar. 7, 1995; for making C7-epi-fluoro see, G. Roth et al, Tetrahedron Letters, Vol 36, pp 1609-1612 (1993); for forming C7 esters and carbonates see, U.S. Pat. No. 5,272,171 issued Dec. 21, 1993 and S. H. Chen et al., Tetrahedron, 49, No. 14, pp 2805-2828 (1993).

In U.S. Pat. No. 5,773,464, TAXOL derivatives containing epoxides at the C₁₀ position are disclosed as antitumor agents. Other C-10 taxane analogs have also appeared in the literature. Taxanes with alkyl substituents at C-10 have been reported in a published PCT patent application WO 9533740. The synthesis of C-10 epi hydroxy or acyloxy compounds is disclosed in PCT application WO 96/03394.Additional C-10 analogs have been reported in Tetrahedron Letters 1995, 36(12), 1985-1988; J. Org. Chem. 1994, 59, 4015-4018 and references therein; K. V. Rao et. al. Journal of Medicinal Chemistry 1995, 38 (17), 3411-3414; J. Kant et. al. Tetrahedron Lett. 1994, 35(31), 5543-5546; WO 9533736; WO 93/02067; U.S. Pat. No. 5,248,796; WO 9415929; and WO 94/15599.

Other relevant TAXOL derivatives include the sulfenamide taxane derivatives described in U.S. Pat. No. 5,821,263. These compounds are charachterized by the C3′ nitrogen bearing one or two sulfur substiuents. These compounds have been useful in the treatment of cancers such as ovarian, breast, lung, gastic, colon, head, neck, melanoma, and leukemia.

U.S. Pat. No. 4,814,470 discusses TAXOL derivatives with hydroxyl or acetyl group at the C10 position and hydroxy or t-butylcarbonyl at C2′ and C3′ positions.

U.S. Pat. No. 5,438,072 discusses TAXOL derivatives with hydroxyl or acetate groups at the C10 position and a C2′ substitutuent of either t-butylcarbonyl or benzoylamino.

U.S. Pat. No. 4,960,790 discusses derivatives of TAXOL which have, at the C2′ and/or C7 position a hydrogen, or the residue of an amino acid selected from the group consisting of alanine, leucine, isoleucine, saline, phenylalanine, proline, lysine, and arginine, or a group of the formula:

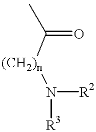

wherein n is an integer of 1 to 3 and R² and R³ are each hydrogen on an alkyl radical having one to three carbon atoms or wherein R² and R³ together with the nitrogen atom to which they are attached form a saturated heterocyclic ring having four to five carbon atoms, with the proviso that at least one of the substituents are not hydrogen.

Other similar water soluble TAXOL derivatives are discussed in U.S. Pat. Nos. 4,942,184, 5,433,364, and in 5,278,324.

Many TAXOL derivatives may also include protecting groups such as, for example, hydroxy protecting groups. “Hydroxy protecting groups” include, but are not limited to, ethers such as methyl, t-butyl, benzyl, p-methoxybenzyl, p-nitrobenzyl, allyl, trityl, methoxymethyl, methoxyethoxymethyl, ethoxyethyl, tetrahydropyranyl, tetrahydrothiopyranyl, dialkylsilylethers, such as dimethylsilyl ether, and trialkylsilyl ethers such as trimethylsilyl ether, triethylsilyl ether, and t-butyldimethylsilyl ether; esters such as benzoyl, acetyl, phenylacetyl, formyl, mono-, di-, and trihaloacetyl such as chloroacetyl, dichloroacetyl, trichloroacetyl, trifluoroacetyl; and carbonates such as methyl, ethyl, 2,2,2-trichloroethyl, allyl, benzyl, and p-nitrophenyl. Additional examples of hydroxy protecting groups may be found in standard reference works such as Greene and Wuts, Protective Groups in Organic Synthesis, 2d Ed., 1991, John Wiley & Sons, and McOmie; and Protective Groups in Organic Chemistry, 1975, Plenum Press. Methods for introducing and removing protecting groups are also found in such textbooks.

### Example 1

### Identification of Sensitivity and Resistance Genes

### Cancer Cell Line Preparation

Sixty cancer cell lines were obtained from the National Cancer Institute Developmental Therapeutics Program (NCI-DTP). Procedures for growing cells and testing compounds have been described previously (Scudiero et al., Cancer Res. 1988, 48:4827-4833; Stinson et al., Anticancer Res.; Myers et al., Electrophoresis 1997, 18:647-653). Cells are plated on day 0 at a density individualized for each cell line so that they will generally be sub-confluent at the end of the assay period. On day 1, a compound is added in the format for a duplicate-well, 5-dose, ten-fold interval dose response study.

No-drug, no-cell and no-growth controls are included. On day 3 the cells are processed for staining with sulforhodamine B (SRB), which reflects the amount of cell mass present at the end of a 48 hour exposure to the test agent. From dose response curves based on the SRB data, various parameters can be determined. The one used in the present study is the GI₅₀, defined as the concentration of compound required to inhibit growth of the cell line by 50%. More precisely, the quantity used in the calculation to be described is the potency measure −log{GI₅₀}.

### Activity Database (A)

Two tables were created: a table consisting of the growth inhibition (GI₅₀) values for 54 of the 60 cell lines and 171 compounds was created from the NCI-DTP in vitro cancer screen database. These were the seed compounds representing the major classes of compounds present in the larger 23,000 compounds database available from the DTP. The seed compounds were selected on the basis of their known mechanism of action and chemical structure. The average potency −log {GI₅₀} was extracted from the flat comma-delimited text files available through the Web at http://www.nci.nih.gov/intra/lmp/jnwbio.html. Missing values were left as a blanks in the data tables.

### Oligonucleotide Array Expression Monitoring Chip

The Affymetrix HUM6000 GeneChip system was used (Affymetrix, Inc.; Santa Clara, Calif.) to measure expression.

The HUM6000 chip design, consisting of 65,000 features each containing 10 million oligonucleotides designed on the basis of sequence data available from GenBank, was employed in the studies described below. The oligonucleotides on the arrays were designed at Affymetrix to cover the complementary strand at the 3′ end of the human genes. About 4000 known fully sequenced human gene cDNA's and more than 2000 human EST's displaying some similarity with known genes characterized in other organisms are represented on a set of four chips. Most genes are represented by 20 overlapping oligonucleotides. A mismatch oligonucleotide is included for each probe design. The sequence of the oligonucleotide probes on the arrays are selected based on a combination of sequence uniqueness-criteria and empirical rules developed at Affymetrix for the selection of oligonucleotides.

### RNA Extraction and Preparation for Hybridization

Double passed polyA RNA was prepared from the cell line pellets (˜10⁸ cells/pellet) using Invitrogen Fast Track 2.0 system.

The isolated polyA RNA (2 μg) was used to synthesize cDNA using Gibco BRL Superscript Choice System cDNA Synthesis Kit. The following modified T7 RNA polymerase promoter -[T]24 primer was used:

5′-GGCCAGTGAATTGTAATACGACTCACTATAGGGAGGCGG-[T]24-3′

To prepare labeled cRNA, double stranded cDNA was passed through a Phase Lock Gel (PLG, 5 Prime-3 Prime, Inc.; Boulder, Colo.) and precipitated with 0.5 vol. of 7.5M NH₄OAc and 2.5 vol. of cold 100% EtOH. The in vitro transcription reaction (IVT) was carried out using T7 RNA polymerase (T7 Megascript System: Ambion; Austin, Tex.) with the following modifications: biotin-11-CTP and biotin-16-UTP (ENZO Diagnostics; Farmingdale, N.Y.) were added to the rNTP cocktail for the IVT reaction. The reaction was incubated for 6 h at 37° C. products were cleaned over a RNeasy Kit (Qiagen; Chatsworth, Calif.). About 45 μg of cRNA was fragmented by incubating at 94° C. for 35 min in 40 mM Tris-Acetate pH 8.1, 100 mM potassium acetate and 30 mM magnesium acetate.

### Array Hybridization and Scanning

Hybridization solutions contained 1.0 M NaCl, 10 mM Tris-HCl (pH 7.6) and 0.005% Triton X-100, and 0.1 mg/ml unlabeled, sonicated herring sperm DNA (Promega). cRNA samples were heated in the hybridization solution to 99° C. for 5 min followed by 45° C. for 5 min before being placed in the hybridization cartridge. Hybridization was carried out at 40° C. for 16 h with mixing on a rotisserie at 60 rpm. Following hybridization, the solutions were removed, the arrays were rinsed with 6×SSPE-T (0.9 M NaCl, 60 mM NaH₂PO₄, 6. mM EDTA, 0.005% Triton X-100 adjusted to pH 7.6), incubated with 6×SSPE-T for 1 hour at 50° C. and then washed with 0.5×SSPE-T at 50° C. for 15 min. Following washing, the hybridized cRNA was flourescently labeled by incubating with 2 μg/ml streptavidine-phycoerythrin (Molecular Probes, Eugene, Oreg.) and 1 mg/ml acetylated BSA (Sigma, St. Louis, Mo.) in 6×SSPE-T at 40° C. for 10 min. Unbound streptavidine-phycoerythrin was removed by rinsing at room temperature prior to scanning. Scanning was done on a specially designed confocal scanner made for Affymetrix by Molecular Dynamics. The excitation source was an argon ion laser and the emission was detected by a photomultiplier tube through a 560 nm longpass filter.

### Quantitative Analysis of Hybridization Patterns and Intensities

Following a quantitative scan of an array, a grid was aligned to the image using the known dimensions of the array and the corner and edge controls regions as markers. The pixels in each region (about 20) were averaged after discarding outliers and pixels near feature boundaries. The image was reduced to a text file containing position, oligonucleotide sequence, ORF or locus name and intensity information. To determine the quantitative RNA abundance, the average of the difference (PM minus MM) for each probe family was calculated (after discarding the maximum, minimum and any outliers beyond three standard deviations from the computed mean).

### Gene Expression Database (E)

A table consisting of the gene expression intensities was created for the 54 cell lines. Inter-chip variability was corrected by adjusting the intensities to the average of the mean of the total signal across the different chips subtypes (A to D).

### Correlation and Cluster Analysis

The various GI₅₀ correlation maps were obtained using a series of mathematic steps described elsewhere (Weinstein et al., Science, 275:343-349 (1997)). Each database (A and E) was treated as a mathematical matrix and the following three steps were applied: (A) each rows of A and E was normalized by its mean and standard deviation; (B) the two matrixes were multiplied using the Pearson correlation function of Microsoft Excel to obtain E.A′ where the prime symbol indicates the matrix transpose; and (C) the rows and the columns were cluster ordered using the Clustal W 1.7 package (Thompson et al., Nucleic Acids Research, 22:4673-4680 (1994)).

### Data Interpretation

Using the Pearson correlation coefficient (r) generated a new matrix (E.A′) of correlation coefficient between genes and compounds. The expression of resistance genes is expected to correlate with negative values of r. The expression of sensitizing genes is expected to correlate with positive values of r. The biological relevance of the correlation was further analyzed for the individual resistance/sensitivity associated genes by verification of the amplitude of variation in the expression (E) and drug response data (A).

### Summary of Data

The sensitivity and resistance genes identified in this Example are summarized in Tables 1, 2a, and 2b . Table 1 provides a summary of the genes whose expression is strongly correlated with sensitivity or resistance to the listed agents. Each row refers to a different gene; each column refers to a different agent. Correlation scores of expression to sensitivity/resistance to an agent are provided at the intersection. Sensitivity to an agent is indicated as a value that ranges from .1 (weak, but statistically identifiable correlation) to 0.9 (strong, statistically significant correlation). Resistance to an agent is indicated as a value that ranges from −0.1 (weak, but statistically identifiable correlation) to −0.9 (strong, statistically significant correlation).

Tables 2a and 2b provide all of the data generated that shows any correlation between sensitivity or resistance to an agent (GI₅₀) and gene expression where there was at least one correlation between gene expression and sensitivity/resistance greater than 0.7 (Table 2a) or less than −0.7 (Table 2b) for the gene. Each row refers to a different gene; each column refers to a different agent. Correlation scores of expression to sensitivity/resistance to an agent are provided at the intersection. Sensitivity to an agent is indicated as a value that ranges from 0.1 (weak, but statistically identifiable correlation) to 0.9 (strong, statistically significant correlation). Resistance to an agent is indicated as a value that ranges from −0.1 (weak, but statistically identifiable correlation) to −0.9 (strong, statistically significant correlation).

### Table 1: Subset Of Clinically Used Compounds

Table 1 provides a summary of the correlation's between gene expression and compound GI₅₀ response across 54 cell lines. The cell lines are listed in Table 5 along with their tissue origin. A high positive Pearson correlation coefficient indicates that the agent tends to be selectively active against the cells that express the gene at a high level (as a measure of transcript abundance). Negative values of the Pearson correlation coefficient indicates that the agent tends to be selectively active against the cells that express the gene in small amount. The columns represent the subset of 26 seed compounds from. the NCI-DTP database routinely used in clinic and organized according to their mode of action.

The rows represent a subset of genes displaying the highest correlation coefficients (r>0.7 or r<−0.7) and clustering apparently according to the mechanism of action of the drugs. Genbank accession numbers for the genes are provided. Some of the genes are human ESTs: in that case the accession number refers to that organism's gene displaying the best homology to the EST translated sequence.

Correlations for Daunorubicin and Deoxydoxorubicin with Human P-glycoprotein are 0.8 and 0.9 respectively.

### Tables 2A and 2B: GI₅₀ Clustered Correlations

Table 2A and 2B provide the correlations between gene expression pattern and compound GI₅₀ response across 54 cell lines that show sensitivity (Table 2A) or resistance (Table 2B). The cell lines are listed in Table 5 along with their tissue origin.

A positive Pearson correlation coefficient indicates that the agent tends to be selectively active against the cells that express the gene at a high level. A negative Pearson correlation coefficient indicates that the agent tends to be selectively active against the cells that express the gene at a low level. The 311 columns represent the subset of 171 seed compounds from the NCI-DTP database organized according to their mode of action whenever this information is available.

Targets are cluster ordered as explained in Materials and Methods.

Table 2A represents the list of genes that have a correlation coefficient of >0.7 with at least one compound (sensitivity genes).

Table 2B represents the list of genes that have a correlation coefficient of <−0.7 with at least one compound (resistance genes).

Tables 6A and 6B provide the correlations between gene expression pattern and compound GI₅₀ response across 46 selected cell lines that show sensitivity (Table 6A) or resistance (Table 6B).

A positive Pearson correlation coefficient indicates that the agent tends to be selectively active against the cells that express the gene at a high level. A negative Pearson correlation coefficient indicates that the agent tends to be selectively active against the cells that express the gene at a low level. The columns represent a subset of 26 seed compounds from the NCI-DTP database routinely used in clinic and organized according to their mode of action.

The rows represent a subset of genes displaying the highest correlation coefficients (r>0.4 in Table 6A (sensitivity genes) or r<−0.4 in Table 6B (resistance genes)) for at least one agent and clustering apparently according to the mechanism of action of the drugs. Genbank accession numbers for the genes are provided. Some of the genes are human ESTs: in that case the accession number refers to that organism's gene displaying the best homology to the EST translated sequence.

The 46 selected cell lines (a subset of the 54 cell lines in Table 5) analyzed to generate the data in Tables 6A and 6B were selected because they do not express the multi-drug resistance gene MDR1 at a high level. By analyzing this subset of cell lines, subtle correlations that may have been masked by the dominance of MDR1 were revealed.

### Compounds, Genes, and Cell Lines

Compounds (C) are identified in Table 3. Genes (G) are identified in Table 4. Cell lines are identified in Table 5.

### Example 2

### Identification of Sensitivity and Resistance Genes In Vivo

In this example, two murine epithelial tumor cell lines were studied. Both of these cell lines were derived from the same parental tumor cell line. The cell lines were obtained through a selection process that involved implanting the parental cell line into mice treated with cyclophosphamide or cisplatin. Tumors which developed in the drug-treated mice were isolated and implanted in successive drug-treated mice until a strongly drug resistant phenotype was achieved. In vitro, the resulting cell lines are sensitive to cyclophosphamide or cisplatin. However, when the same cell lines are implanted into a mouse, one of the cell lines, CTX, selected for resistance to cyclophosphamide, is resistant to cyclophosphamide. The other cell line, CDDP, selected for resistance to cisplatin, is resistant to cisplatin.

Each tumor cell line was grown in vivo and gene expression was measured using the Mu6500 murine gene chip system available from Affymetrix, Inc. (Santa Clara, Calif.). Expression assays were done in triplicate using the CTX cell line, the CDDP cell line, and the parental cell line. The results of this analysis are presented in Tables 7A, 7B, 7C, and 7D.

Table 7A lists the genes that are expressed at a higher level in the cyclophosphamide resistant tumor cell line, CTX, than in the parental cell line.

Table 7B lists the genes that are expressed at a lower level in the cyclophosphamide resistant tumor cell line, CTX, than in the parental tumor cell line.

Table 7C lists the genes that are expressed at a higher level in the cisplatin resistant tumor cell line, CDDP, than in the parental tumor cell line.

Table 7D lists the genes that are expressed at a lower level in the cisplatin resistant cell line, CDDP, than in the parental tumor cell line.

In Tables 7A, 7B, 7C, and 7D, column one is the Genbank accession number of the gene (or EST), columns two is the description of the gene (or EST), columns three to five are the expression levels of the indicated gene in the CTX or CDDP tumor cell line in each of three experiments, columns six to eight are the expression levels of the indicated gene in the parental cell line in each of three experiments, column nine is the average expression level in the CTX or CDDP tumor cell line, column ten is the average expression level in the parent cell line, and column eleven is ratio of the average expression in the CTX or CDDP tumor cell line to the average expression in the parental cell line.

### Example 3

### Examples of Sensitivity/Resistance Assays

This example and the next example describe sensitivity assays and resistance assays for a number of agents based on the data provided in Table 1. It should be recognized that the gene descriptions refer to sequence immobilized on an Affymetrix HUM6000 gene chip and that the names associated with the sequences may not be the actual names of the genes that are hybridizing to the bound probe. Accordingly, to perform the assays described herein, a sequence corresponding to the Affymetrix probe is used as the marker of expression of a resistance or sensitivity gene.

It should also be recognized that, although the examples employ data provided in Table 1, the data provided in Tables 2a and 2b provide additional suitable genes for use in sensitivity assays and resistance assays.

To determine sensitivity to 5-fluorouracil, the sensitivity genes are selected from the group of genes having a relatively high positive Pearson correlation coefficient for 5-fluorouracil, e.g., the group comprising: human follistatin gene, PTB-associated splicing factor, human fibroblast growth factor receptor (FGFr), CCAAT-binding transcription factor I subunit A.

To determine resistance to 5-fluorouracil, the resistance genes are selected from the group of genes having a relatively high negative Pearson correlation coefficient for 5-fluorouracil, e.g., the group comprising: human mRNA for pro-alpha 1 (11) collagen (3′ end C-term. triple helical and C-terminal non-helical domain), H. sapiens mRNA for red cell anion exchanger (EPB3,AEI, Band 3) 3′ non-coding region, choline kinase, monocyte chemoattractant protein 1 receptor, blood group rh(d) polypeptide, lactotransferrin precursor (human homolog of Bos taurus), brain calcium channel bii-2 protein (human homolog of Oryctolagus cuniculus), delta-(1-alpha-aminoadipyl)-1-cysteinyl-d-valine synthetase (human homolog of Penicillium chrysogenum), interleukin-2 receptor beta chain precursor, probable nuclear antigen (human homolog of Pseudorabies virus), ya31c03.s2 homo sapiens cdna clone 62212, and atp synthase a chain (human homolog of Trypanosoma brucei brucei).

To determine sensitivity to TAXOL, the sensitivity genes are selected from the group of genes having a relatively high positive Pearson correlation coefficient for TAXOL, e.g., the group comprising: Protein 2, interleukin 6, and collagen.

To determine resistance to TAXOL, the resistance genes are selected from the group of genes having a relatively high negative Pearson correlation coefficient for TAXOL, e.g., the group comprising: H. sapiens tropomyosin isoform, calpactin I light chain, complement c3 precursor, human dd96 mRna, leukemia inhibitory factor receptor precursor (human homolog of Mus musculus), myosin light chain alkali, smooth-muscle isoform, beta galactosidase-related protein precursor, cell cycle arrest protein bub2 (human homolog of Saccharomyces cerevisiae).

To determine sensitivity to BCNU, the sensitivity genes are selected from the group of genes having a relatively high positive Pearson correlation coefficient for BCNU, e.g., the group comprising: Human SEF2-1A protein (5′ end), protein 2, laminin beta 1 chain, and CCAAT-binding transcription factor 1 subunit A.

To determine resistance BCNU, the resistance genes are selected from the group of genes having a relatively high negative Pearson correlation coefficient for BCNU, e.g., the group comprising: human giant larva homolog, cell cycle arrest protein BUB2 (human homolog), histone H2A.2 and choline kinase.

To determine sensitivity to carboplatin, the sensitivity genes are selected from the group of genes having a relatively high positive Pearson correlation coefficient for carboplatin, e.g., the group comprising: BMP-2B, Annexin V (human homolog of Gallus gallus), human giant larva homolog, B-cell lymphoma-3 encoded protein, ryanodine receptor, protein tyrosine phosphotase alpha precursor, tubulin alpha chain (human homolog of Lytechinus pictus), and peripheral myelin protein 22.

To determine resistance to carboplatin, the sensitivity genes are selected from the group of genes having a relatively high positive Pearson correlation coefficient for carboplatin, e.g., the group comprising: protein 2, collagen, and follistatin gene exon 6.

### Example 4

### The Identification of Therapeutic and Drug Screening Targets

The expression of resistance genes is expected to correlate with a negative Pearson correlation coefficient. The following genes have been shown to have a resistance phenotype. These genes are potential therapeutic targets. They are also potentially useful drug screening targets.

The expression of the human choline kinase gene has shown a negative Pearson correlation to the agents triethylenemelamine, chlorambucil, uracil nitrogen mustard, pipobroman, vinblastine sulfate, bleomycin, hydroxyurea, 5-FUDR, and 2-dexoycoformycin. Thus, cells expressing the human choline kinase gene at a relatively high level would be expected to be resistant to these agents.

The expression of the human p300 gene has shown a negative Pearson correlation to the agents doxorubicin, daunorubicin (daunomycin), VM-26 (teniposide), DHAD (mitoxantrone), vinblastine sulfate, and actinomycin D. Thus, cells expressing the human p300 gene at a relatively high level would be expected to be resistant to these agents.

The expression of the human giant larvae homolog gene has shown a negative Pearson correlation to the agents, agents chlorambucil, uracil nitrogen mustard, daunorubicin (daunomycin), BCNU, cisplatin, CBDCA (carboplatin), thioguanine, cytosine arabinoside, fludarabine phosphate, topotecan, and bleomycin. Thus, cells expressing the human giant larvae homolog gene at a relatively high level would be expected to be resistant to these agents.

The expression of the human mixed lineage kinasel gene has shown a negative Pearson correlation to the agents, doxorubicin, daunorubicin (daunomycin), VM-26 (teniposide), DHAD (mitoxantrone), vinblastine sulfate, actinomycin D, topotecan, and bleomycin. Thus, cells expressing the human mixed lineage kinasel gene at a relatively high level would be expected to be resistant to these agents.

The expression of the human 9112 (14-3-3 sigma) gene has shown a negative Pearson correlation to the agents, BCNU, busulfan, chlorambucil, melphalan, nitrogen mustard, cisplatin, vinblastine sulfate, daunorubicin, deoxydoxorubicin, doxorubicin, VM-26 (teniposide), DHAD (mitoxantrone), VP-16 (etoposide), and topotecan. Thus, cells expressing the human 9112 (14-3-3 sigma) gene at a relatively high level would be expected to be resistant to these agents.

The expression of the human phospholipid hydroperoxide gluthatione peroxidase gene has shown a negative Pearson correlation to the agents, vincristine sulfate, daunorubicin, deoxydoxorubicin, doxorubicin, and VM-26 (teniposide). Thus, cells expressing the human human phospholipid hydroperoxide gluthatione peroxidase gene at a relatively high level would be expected to be resistant to these agents.

The expression of the human alpha-7-thiol proteinase inhibitor gene has shown a negative Pearson correlation to the agents, BCNU, busulfan, chlorambucil, melphalan, tetraplatin platinum, hydroxyurea, daunorubicin, deoxydoxorubicin, VM-26 (teniposide), and VP-16 (etoposide). Thus, cells expressing the human alpha-7-thiol proteinase inhibitor gene at a relatively high level would be expected to be resistant to these agents.

The expression of the clathrin coat assembly protein AP47 gene has shown a negative Pearson correlation to the agents, BCNU, busulfan, chlorambucil, melphalan, nitrogen mustard, cisplatin, TAXOL, vinblastine sulfate, methotrexate, daunorubicin, deoxydoxorubicin, doxorubicin, VM-26 (teniposide), DHAD (mitoxantrone), VP-16 (etoposide), and topotecan. Thus, cells expressing the clathrin coat assembly protein AP47 gene at a relatively high level would be expected to be resistant to these agents.

By examining the expression of one or more of the identified resistance genes in a sample of cancer cells, it is possible to determine which therapeutic agent(s), or combination of agents, to use as the appropriate treatment agents. For example, if the expression of human alpha-7-thiol proteinase inhibitor gene in a sample of cancer cells was found to be relatively high, it would suggest that BCNU, busulfan, chlorambucil, melphalan, tetraplatin platinum, hydroxyurea, daunorubicin, deoxydoxorubicin, VM-26 (teniposide), and VP-16 (etoposide) would be relatively ineffective and another course of treatment may be pursued.

By examining the expression of one or more of the identified resistance genes in a sample of cancer cells taken from a patient during the course of therapeutic treatment, it is possible to determine whether the therapeutic agent is continuing to work or whether the cancer has become resistant (refractory) to the treatment protocol. For example, a cancer patient receiving a treatment of vinblastine sulfate would have cancer cells removed and monitored for the expression of the human 9112 (14-3-3 sigma) gene. If the human 9112 (14-3-3 sigma) gene transcripts remain unelevated, the treatment with vinblastine would continue. However, an increase in human 9112 (14-3-3 sigma) gene expression would suggest that the cancer has become resistant to vinblastine sulfate and another chemotherapy protocol would be initiated to treat the patient.

Importantly, these determinations can be made on a patient by patient basis or on an agent by agent (or combinations of agents). Thus, one can determine whether or not a particular therapeutic treatment is likely to benefit a particular patient or group/class of patients, or whether a particular treatment should be continued.

The identified resistance genes further provide previously unknown or unrecognized targets for the development of anti-cancer agents, such as chemotherapeutic compounds, and can be used as targets in developing single agent treatment as well as combinations of agents for the treatment of cancer. For example, increased expression of the human choline kinase gene has been implicated in the resistance of cancer cells to triethylenemelamine, chlorambucil, uracil nitrogen mustard, pipobroman, vinblastine sulfate, bleomycin, hydroxyurea, 5-FUDR, and 2-dexoycoformycin. Modulators of human choline kinase expression or activity could be used in conjunction with agents triethylenemelamine, chlorambucil, uracil nitrogen mustard, pipobroman, vinblastine sulfate, bleomycin, hydroxyurea, 5-FUDR, and 2-dexoycoformycin as combination therapy for drug resistant tumors.

### Example 5

### Identification of TAXOL Sensitivity and Resistance Genes

Described below are three different studies designed to identify genes that are differentially expressed in TAXOL sensitive and TAXOL resistant cancer cells.

In the first study, nucleic acid arrays were used to determine the level of expression of approximately 6500 nucleic acid sequences in selected relatively highly TAXOL resistant and relatively highly TAXOL sensitive solid tumor cell lines from the NCI 60 cancer cell line series. This analysis led to the identification of genes that are relatively highly expressed in TAXOL resistant cancer cell lines (Tables 8, 9A, 9B, 9C, and 9D) and genes that are relatively highly expressed in relatively highly TAXOL sensitive cancer cells lines (Table 8B).

In the second study, nucleic acid arrays were used to determine the level of expression of approximately 6500 nucleic acid sequences in a relatively TAXOL resistant human mammary epithelial cell primary cell line (HMEC) and in a relatively TAXOL sensitive breast cancer cell line (MDA-435) in the presence of TAXOL. This analysis led to the identification of genes that are relatively highly expressed in the TAXOL resistant human mammary epithelial cell primary cell line compared to the relatively TAXOL sensitive breast cancer cell line (Table 10 A) and genes that are relatively highly expressed in the relatively TAXOL sensitive breast cancer cell line compared to the relatively TAXOL resistant human mammary epithelial cell primary cell line (Table 10B). In the third study, nucleic acid arrays were used to determine the level of expression of approximately___nucleic acid sequences in breast cancer clinical samples obtained from patients whose breast cancer appeared to respond to TAXOL/cisplatin combination therapy over an initial six month treatment period (“TAXOL/cisplatin sensitive clinical samples”) and breast cancer clinical samples obtained from patients whose breast cancer appeared to respond poorly to TAXOL/cisplatin combined therapy over and initial six month treatment period (“TAXOL/cisplatin resistant clinical samples”). This analysis led to the identification of genes that are expressed at a relatively high level in the TAXOL/cisplatin resistant clinical samples compared to the TAXOL/cisplatin sensitive clinical samples (Table 11A) and genes that are expressed at a relatively low level in the TAXOL/cisplatin resistant clinical samples compared to the TAXOL/cisplatin sensitive clinical samples (Table 11B).

### Differential Expression of Genes in TAXOL Resistant and TAXOL Sensitive Cancer Cell Lines

In this study, solid tumor cell lines obtained from the National Cancer Institute Developmental Therapeutics Program (NCI-DTP) were studied to identify genes associated with resistance or sensitivity to TAXOL. In one phase of this study, nine relatively highly TAXOL resistant tumor cell lines of various types and nine relatively highly TAXOL sensitive cell lines of various types were studied (Tables 8A and 8B). In another phase of this study, relatively TAXOL resistant and relatively TAXOL sensitive melanoma cell lines (Table 9A), breast cancer cell lines (Table 9B), colon cancer cell lines (Table 9C) and ovarian cancer cell lines (Table 9D) were studied.

Procedures for growing cells have been described previously (Scudiero et al., Cancer Res. 1988, 48:4827-4833; Stinson et al., Anticancer Res.; Myers et al., Electrophoresis 1997, 18:647-653).

The Affymetrix HUM6000 GeneChip system (Santa Clara, Calif.) was used to measure expression of approximately 6500 nucleic acid sequences in the selected cell lines. The cRNA used for expression analysis was prepared as follows. First, double passed polyA RNA was prepared from the cell line pellets (˜10⁸ cells/pellet) using Invitrogen Fast Track 2.0 system. Next, cDNA was prepared from 2 μg of polyA RNA using Gibco BRL Superscript Choice System cDNA Synthesis Kit. The following modified T7 RNA polymerase promoter -[T]24 primer was used:

5′-GGCCAGTGAATTGTAATACGACTCACTATAGGGAGGCGG-[T]24-3′

To prepare cRNA, double stranded cDNA was passed through a Phase Lock Gel (PLG, 5 Prime-3 Prime, Inc.; Boulder, Colo.) and precipitated with 0.5 vol of 7.5M NH₄OAc and 2.5 vol of cold 100% ethanol. In vitro transcription (IVT) was carried out using T7 RNA polymerase (T7 Megascript System: Ambion; Austin, Tex.) with the following modifications: biotin-11-CTP and biotin-16-UTP (ENZO Diagnostics; Farmingdale, N.Y.) were added to the rNTP cocktail for the IVT reaction. The reaction was incubated for 6 h at 37° C. and the products were cleaned using an RNeasy Kit (Qiagen; Chatsworth, Calif.). About 45 μg of cRNA was fragmented by incubating at 94° C. for 35 min in 40 mM Tris-Acetate pH 8.1, 100 mM potassium acetate and 30 mM magnesium acetate.

Hybridization solutions contained 1.0 M NaCl, 10 mM Tris-HCl (pH 7.6), 0.005% Triton X-100, and 0.1 mg/ml unlabeled, sonicated herring sperm DNA (Promega). The cRNA samples were heated in the hybridization solution to 99° C. for 5 min followed by 45° C. for 5 min before being placed in the hybridization cartridge. Hybridization was carried out at 40° C. for 16 hours with mixing on a rotisserie at 60 rpm. Following hybridization, the solutions were removed and the arrays were rinsed with 6×SSPE-T (0.9 M NaCl, 60 mM NaH₂PO₄, 6 mM EDTA, 0.005% Triton X-100 adjusted to pH 7.6). Next, the arrays were incubated with 6X SSPE-T for 1 hour at 50° C. and then washed with 0.5×SSPE-T at 50° C. for 15 min. Following washing, the hybridized cRNA was flourescently labeled by incubating with 2 μg/ml streptavidin-phycoerythrin (Molecular Probes, Eugene, Oreg.) and 1 mg/ml acetylated BSA (Sigma, St. Louis, Mo.) in 6×SSPE-T at 40° C. for 10 min. Unbound streptavidin-phycoerythrin was removed by rinsing at room temperature prior to scanning. Scanning was done on a specially designed confocal scanner made for Affymetrix by Molecular Dynamics. The excitation source was an argon ion laser and the emission was detected by a photomultiplier tube through a 560 nm longpass filter.

Following a quantitative scan of an array, a grid was aligned to the image using the known dimensions of the array and the comer and edge controls regions as markers. The pixels in each region (about 20) were averaged after discarding outliers and pixels near feature boundaries. The image was reduced to a text file containing position, oligonucleotide sequence, ORF or locus name and intensity information. To determine the quantitative RNA abundance, the average of the difference (PM minus MM) for each probe family was calculated (after discarding the maximum, minimum and any outliers beyond three standard deviations from the computed mean).

Table 8A lists genes that are relatively highly expressed in the selected relatively highly TAXOL resistant cancer cell lines compared to the selected relatively highly TAXOL sensitive cell lines. Table 8B lists genes that are relatively highly expressed in the selected relatively TAXOL sensitive cancer cell lines compared to the selected relatively TAXOL resistant cancer cell lines. In Tables 8A and 8B, the first column presents a description of the gene (or EST), the second column presents the Genbank accession number of the gene (or EST), the third through eleventh columns present expression data for the indicated genes in the indicated relatively highly TAXOL resistant cancer cell lines (EKVX, HOP92, HCT15, MALME-3M, SK-MEL-26, OVCAR4,ACHN, MCF7-ADR, and T-47D, respectively), and the twelfth through twentieth columns present expression data for the indicated genes in the indicated relatively highly TAXOL sensitive cell lines (NCI-H460, NCI-H522, HT29, SKMEL2, SKMEL5, OVCAR-3, SN12C, MCF7, and MDA-MB-435, respectively).

Table 9A lists genes that are relatively highly expressed in selected relatively TAXOL resistant melanoma cell lines compared to selected relatively TAXOL sensitive melanoma cell lines. In Table 9A, the first column presents a description of the gene (or EST), the second column presents the Genbank accession number of the gene (or EST), the third and fourth columns present expression data for the indicated genes in the indicated relatively TAXOL resistant melanoma cell lines (MALME-3M and SK-MEL-28, respectively), and the fifth and sixth columns present expression data for the indicated genes in the indicated relatively TAXOL sensitive melanoma cell lines (LOX1MV1 and SKMEL5, respectively).

Table 9B lists genes that are relatively highly expressed in selected relatively TAXOL resistant breast cancer cell lines compared to selected relatively TAXOL sensitive breast cancer cell lines. In Table 9B, the first column presents a description of the gene (or EST), the second column presents the Genbank accession number of the gene (or EST), the third and fourth columns present expression data for the indicated genes in the indicated relatively TAXOL resistant breast cancer cell lines (T-47D and MDAM231, respectively), and the fifth through eighth columns present expression data for the indicated genes in the indicated relatively TAXOL sensitive breast cancer cell lines (MCF7, MDA-MB-435, HS578T, and MDAN, respectively).

Table 9C lists genes that are relatively highly expressed in selected relatively TAXOL resistant colon cancer cell lines compared to selected relatively TAXOL sensitive colon cancer cell lines. In Table 9C, the first column presents a description of the gene (or EST), the second column presents the Genbank accession number of the gene (or EST), the third and fourth columns present expression data for the indicated genes in the indicated relatively TAXOL resistant colon cancer cell lines (DLD1 and HCT15, respectively), and the fifth through tenth columns present expression data for the indicated genes in the indicated relatively TAXOL sensitive colon cancer cell lines (HCT116, HCC2998, COL0205, SW620, KM12, and HT-29, respectively).

Table 9D lists genes that are relatively highly expressed in selected relatively TAXOL resistant ovarian cancer cell lines compared to selected relatively TAXOL sensitive ovarian cancer cell lines. In Table 9D, the first column presents a description of the gene (or EST), the second column presents the Genbank accession number of the gene (or EST), the third and fourth columns present expression data for the indicated genes in the indicated relatively TAXOL resistant ovarian cancer cell lines (OVCAR4 and OVCAR5, respectively), and the fifth through seventh columns present expression data for the indicated genes in the indicated relatively TAXOL sensitive ovarian cancer cell lines (OVCAR82, OVCAR3, and IGROV, respectively).

The genes listed in Tables 8A, 9A, 9B, 9C, and 9D are resistance genes with respect to TAXOL. Thus, they can be used, for example, to determine whether or not TAXOL or a related therapeutic agent can be used to reduce the growth of cancer cells. Such a method can include the following steps: a) obtaining a sample of cancer cells; b) determining whether the cancer cells express one or more of the genes listed in Table 8A and Tables 9A-9D; and c) identifying that TAXOL or a related therapeutic agent cannot be used to reduce the growth of the cancer cells when one or more of the listed genes is expressed by the cancer cells.

The genes listed in Table 8B are sensitivity genes with respect to TAXOL. Thus, they can be used, for example, to determine whether or not TAXOL or a related therapeutic agent can be used to reduce the growth of cancer cells. Such a method can include the following steps: a) obtaining a sample of cancer cells; b) determining whether the cancer cells express one or more of the genes listed in Table 8B; and c) identifying that TAXOL or a related therapeutic agent can be used to reduce the growth of the cancer cells when one or more of the listed genes is expressed by the cancer cells.

The resistance and sensitivity genes listed in Tables 8A, 8B, 9A, 9B, 9C, and 9D can be used in other methods described herein. For example, they can be used to determine whether a selected TAXOL related compound might be useful for the treatment of a cancer. Such a method can include the following steps: a) obtaining a sample of cancer cells; b) exposing the cancer cells to one or more test agents; c) determining the level of expression in the cancer cells of one or more genes resistance genes selected from Tables 8A, 9A, 9B, 9C, and 9D in the sample exposed to the agent and in a sample of cancer cells that is not exposed to the agent; and d) identifying that an agent cannot be used to reduce the growth of said cancer cells when the expression of one or more of the genes is increased in the presence of the agent.

### Differential Expression of Genes in TAXOL Sensitive and TAXOL Resistant Breast Cells

In this study, nucleic acid arrays were used to determine the level of expression of approximately 6500 nucleic acid sequences in a relatively TAXOL sensitive human mammary epithelial cell primary cell lines (HMEC) and in a relatively TAXOL sensitive breast cancer cell line (MDA-435) in the presence and absence of TAXOL. This analysis led to the identification of genes that are relatively highly expressed in the relatively TAXOL resistant human mammary epithelial cell primary cell line compared to the relatively TAXOL sensitive breast cancer cell line (Table 10A) and genes that are relatively highly expressed in the relatively TAXOL sensitive breast cancer cell line compared to the relatively TAXOL resistant human mammary epithelial cell primary cell line (Table 10B).

The HMEC cells were pooled cells from three individuals. Gene expression in HMEC cells and MDA-435 cells was measured in the presence and absence of TAXOL. The HMEC cells were exposed to 100 nm TAXOL for 12 hours prior to isolation of mRNA for expression analysis. The MDA-435 cells were exposed to 100 nm TAXOL for 12 hours prior to isolation of mRNA for expression analysis. Gene expression was measured as described above for the first study of TAXOL resistant and TAXOL sensitive cell lines.

Table 10A lists genes that are relatively highly expressed in the relatively TAXOL resistant HMEC cells compared to the relatively TAXOL sensitive MDA-435 cell line. Table 10B lists genes that are relatively highly expressed in the relatively TAXOL sensitive MDA-435 cell line compared to the relatively TAXOL resistant HMEC cells. In Tables 10A and 10B, the first column presents a description of the gene (or EST), the second column presents the Genbank accession number of the gene (or EST), the third and fourth columns present expression data for the indicated genes in HMEC cells in the presence of TAXOL and absence of TAXOL respectively, and the fifth and sixth columns present expression data for the indicated gene in MDA-435 cells in the presence and absence of TAXOL respectively.

The genes listed in Table 10A are resistance genes with respect to TAXOL. The genes in Table 10B are sensitivity genes with respect to TAXOL. Thus, the genes listed in Tables 10A and 10B can be used in the methods of the invention in the same manner as other resistance and sensitivity genes described herein.

### Differential Expression of Genes in Responsive and Non-Responsive Ovarian Cancer

In this third study, nucleic acid arrays were used to determine the level of expression of approximately 20,000 nucleic acid sequences in clinical samples obtained from patients whose ovarian cancer appeared to respond to TAXOL/cisplatin combination therapy over an initial six month treatment period (“TAXOL/cisplatin sensitive clinical samples”) and in clinical samples obtained from patients whose ovarian cancer appeared to respond poorly to TAXOL/cisplatin combined therapy over an initial six month treatment period (“TAXOL/cisplatin resistant clinical samples”). This analysis led to the identification of genes that are relatively highly expressed in the TAXOL/cisplatin resistant clinical samples compared to the TAXOL/cisplatin sensitive clinical samples (Table 11A) and genes that are expressed at relatively low level in the TAXOL/cisplatin resistant clinical samples compared to the TAXOL/cisplatin sensitive clinical samples (Table 11B).

The clinical samples were obtained from patients undergoing breast cancer therapy at the Mayo Clinic (Rochester, Minn.). Gene expression was measured as described above for the first study of TAXOL resistant and TAXOL sensitive cell lines except that a proprietary nucleic acid array was used to measure expression.

Table 11A lists genes that are relatively expressed at a relatively high level in the TAXOL/cisplatin resistant clinical samples compared to the TAXOL/cisplatin sensitive clinical samples. Table 11B lists genes that are expressed at a relatively low level in TAXOL/cisplatin resistant clinical samples compared to the TAXOL/cisplatin sensitive clinical samples.

In Tables 11A and 11B, the first column presents a description of the gene (or EST), the second column presents the Unigene accession number of the gene (or EST), the third through sixth columns present expression data for the indicated genes in the indicated TAXOL/cisplatin resistant clinical samples (OV3 (a and b), OV72 (a and b), respectively), and the seventh and eighth columns present expression data for the indicated genes in the indicated TAXOL/cisplatin sensitive clinical samples (OV143 (a and b), respectively).

The genes listed in Table 11A are resistance genes with respect to TAXOL and/or cisplatin. The genes in Table 11B are sensitivity genes with respect to TAXOL and/or cisplatin. Thus, the genes listed in Tables 11A and 11B can be used in the methods of the invention in the same manner as other resistance and sensitivity genes described herein.

### Other Embodiments

The present invention is not to be limited in scope by the specific embodiments described that are intended as single illustrations of individual aspects of the invention and functionally equivalent methods and components are within the scope of the invention, in addition to those shown and described herein will become apparent to those skilled in the art from the foregoing description and accompanying drawings. Such modifications are intended to fall within the scope of the appended claims.

All references cited herein, including journal articles and patents, are expressly incorporated by reference.

What is claimed is:

1. A method for determining whether an agent can be used to reduce the growth of cancer cells, comprising the steps of:
a) obtaining a sample of cancer cells;
b) determining whether said cancer cells express one or more genes selected from the group consisting of the sensitivity genes identified in Tables 8B, 10B, and 11B; and
c) identifying that an agent can be used to reduce the growth of said cancer cells when one or more of said genes is expressed by said cancer cells.

2. A method for determining whether an agent cannot be used to reduce the growth of cancer cells, comprising the steps of:
a) obtaining a sample of cancer cells;
b) determining whether said cancer cells express one or more genes selected from the group consisting of the sensitivity genes identified in Tables 8B, 10B, and 11B; and
c) identifying that an agent cannot be used to reduce the growth of said cancer cells when one or more of said genes is not expressed by said cancer cells.

3. A method for determining whether an agent cannot be used to reduce the growth of cancer cells, comprising the steps of:
a) obtaining a sample of cancer cells;
b) determining whether said cancer cells express one or more genes selected from the group consisting of the resistance genes identified in Tables 8A, 9A, 9B, 9C, 9D, 10A, and 11A; and
c) identifying that an agent cannot be used to reduce the growth of said cancer cells when one or more of said genes is expressed by said cancer cells.

4. The method of claim 1, wherein said level of expression is determined by detecting the amount of mRNA that is encoded by said one or more genes present in said sample.

5. The method of claim 2, wherein said level of expression is determined by detecting the amount of mRNA that is encoded by said one or more genes present in said sample.

6. The method of claim 3, wherein said level of expression is determined by detecting the amount of mRNA that is encoded by said one or more genes present in said sample.

7. The method of claim 1, wherein said level of expression is determined by detecting the amount of protein that is encoded by said one or more genes present in said sample.

8. The method of claim 2, wherein said level of expression is determined by detecting the amount of protein present that is encoded by said one or more genes present in said sample.

9. The method of claim 3, wherein said level of expression is determined by detecting the amount of protein present that is encoded by said one or more genes present in said sample.

10. The method of claim 1, wherein said cancer cells are selected from the group consisting of cancer cell lines and cancer cells obtained from a patient.

11. The method of claim 2, wherein said cancer cells are selected from the group consisting of cancer cell lines and cancer cells obtained from a patient.

12. The method of claim 3, wherein said cancer cells are selected from the group consisting of cancer cell lines and cancer cells obtained from a patient.

13. The method of claim 1, wherein said agent is a chemotherapeutic compound.

14. The method of claim 2, wherein said agent is a chemotherapeutic compound

15. The method of claim 3, wherein said agent is a chemotherapeutic compound.

16. A method for determining whether an agent can be used to reduce the growth of cancer cells, comprising the steps of:
a) obtaining a sample of cancer cells;
b) exposing the cancer cell to one or more test agents;
c) determining the level of expression in the cancer cells of one or more genes selected from the group consisting of the sensitivity genes identified in Tables 8B, 10B, and 11B in the sample exposed to the agent and in a sample of cancer cells that is not exposed to the agent; and
d) identifying that an agent can be used to reduce the growth of said cancer cells when the expression of one or more of said genes is increased in the presence of said agent.

17. A method for determining whether an agent be used to reduce the growth of cancer cells, comprising the steps of:
a) obtaining a sample of cancer cells;
b) exposing the cancer cell to one or more test agents;
c) determining the level of expression in the cancer cells of one or more genes selected from the group consisting of the sensitivity genes identified in Tables 8B, 10B, and 11B in the sample exposed to the agent and in a sample of cancer cells that is not exposed to the agent; and
d) identifying that an agent cannot be used to reduce the growth of said cancer cells when the expression of one or more of said genes is not increased in the presence of said agent.

18. A method for determining whether an agent cannot be used to reduce the growth of cancer cells, comprising the steps of:
a) obtaining a sample of cancer cells;
b) exposing the cancer cell to one or more test agents;
c) determining the level of expression in the cancer cells of one or more genes selected from the group consisting of the resistance genes identified in Tables 8A, 9A, 9B, 9C, 9D, 10A, and 11A in the sample exposed to the agent and in a sample of cancer cells that is not exposed to the agent; and
d) identifying that an agent cannot be used to reduce the growth of said cancer cells when the expression of one or more of said genes is increased in the presence of said agent.

19. The method of claim 16, wherein said level of expression is determined by detecting the amount of mRNA that is encoded by said one or more genes present in said sample.

20. The method of claim 17, wherein said level of expression is determined by detecting the amount of mRNA that is encoded by said one or more genes present in said sample.

21. The method of claim 18, wherein said level of expression is determined by detecting the amount of mRNA that is encoded by said one or more genes present in said sample.

22. The method of claim 16, wherein said level of expression is determined by detecting the amount of protein that is encoded by said one or more genes present in said sample.

23. The method of claim 17, wherein said level of expression is determined by detecting the amount of protein present that is encoded by said one or more genes present in said sample.

24. The method of claim 18, wherein said level of expression is determined by detecting the amount of protein present that is encoded by said one or more genes present in said sample.

25. The method of claim 16, wherein said cancer cells are selected from the group consisting of cancer cell lines and cancer cells obtained from a patient.

26. The method of claim 17, wherein said cancer cells are selected from the group consisting of cancer cell lines and cancer cells obtained from a patient.

27. The method of claim 18, wherein said cancer cells are selected from the group consisting of cancer cell lines and cancer cells obtained from a patient.

28. The method of claim 16, wherein said agent is a chemotherapeutic compound.

29. The method of claim 17, wherein said agent is a chemotherapeutic compound.

30. The method of claim 18, wherein said agent is a chemotherapeutic compound.

31. A method for determining whether treatment with a chemotherapeutic compound should be continued in a cancer patient, comprising the steps of:
a) obtaining two or more samples comprising cancer cells from a patient during the course of chemotherapeutic compound treatment;
b) determining the level of expression in the cancer cells of one or more genes selected from the group consisting of the sensitivity genes identified in Tables 8B, 10B, and 11B in the two or more samples; and
c) continuing treatment when the expression level of one or more of said genes does not decrease during the course of treatment.

32. A method for determining whether treatment with a chemotherapeutic compound should be continued in a cancer patient, comprising the steps of:
a) obtaining two or more samples comprising cancer cells from a patient during the course of chemotherapeutic compound treatment;
b) determining the level of expression in the cancer cells of one or more genes selected from the group consisting of the resistance genes identified in Tables 8A, 9A, 9B, 9C, 9D, 10A, and 11A in the two or more samples; and
c) discontinuing treatment when the expression level of one or more of said genes increases during the course of treatment.

33. A method for determining whether treatment with a chemotherapeutic compound should be continued in a cancer patient, comprising the steps of:
a) obtaining two or more samples comprising cancer cells from a patient during the course of chemotherapeutic compound treatment;
b) determining the level of expression in the cancer cells of one or more genes selected from the group consisting of the resistance genes identified in Tables 8A, 9A, 9B, 9C, 9D, 10A, and 11A in the two or more samples; and
c) continuing treatment when the expression level of one or more of said genes does not increase during the course of treatment.

34. The method of claim 31, wherein said level of expression is determined by detecting the amount of mRNA that is encoded by said one or more genes present in said sample.

35. The method of claim 32, wherein said level of expression is determined by detecting the amount of mRNA that is encoded by said one or more genes present in said sample

36. The method of claim 33, wherein said level of expression is determined by detecting the amount of mRNA that is encoded by said one or more genes present in said sample.

37. The method of claim 31, wherein said level of expression is determined by detecting the amount of protein that is encoded by said one or more genes present in said sample.

38. The method of claim 32, wherein said level of expression is determined by detecting the amount of protein present that is encoded by said one or more genes present in said sample.

39. The method of claim 33, wherein said level of expression is determined by detecting the amount of protein present that is encoded by said one or more genes present in said sample.

40. A method for reducing the growth rate of cancer cells in a patient, comprising the step of administering to a patient suffering from cancer an agent identified using the method of claim 1 as being able to reduce the rate of growth of said cancer cells.
